SEQUENCE LISTING (24 sequences)

SEQ ID NO: 1 - DNA, 27 bp, lentivirus
gaattcaacgcgcggtgaccctcgagg

SEQ ID NO: 2 - DNA, 70 bp, lentivirus
asaaaaaaccaaggcttataacttcgtataatttatactatacgaagttataattactttacagttaccc

SEQ ID NO: 3 - DNA, 77 bp, lentivirus
aattcgagaggcggccgcataacttcgtatagtataaattatacgaagttataagccttgttaacgcgcggtgaccc

SEQ ID NO: 4 - DNA, 77 bp, lentivirus
tcgagggtcaccgcgcgttaacaaggcttataacttcgtataatttatactatacgaagttatgcggccgcctctcg

SEQ ID NO: 5 - DNA, 55 bp, Artificial
  synthetic construct Firefly and Renilla luciferase
tgagctgtttctgaggagccttcaagagaggctcctcagaaacagctcttttttc

SEQ ID NO: 6 - DNA, 59 bp, Artificial
  Synthetic construct Firefly and Renilla luciferase
tcgagaaaaaagctggataatgccaggcagtctcttgaactgcctggcattatccagca

SEQ ID NO: 7 - DNA, 34 bp, Artificial
  Synthetic construct
ataacttcgtatagtataaattatacgaagttat

SEQ ID NO: 8 - DNA, 34 bp, mus musculus
ataacttcgtatagcatacattatacgaagttat

SEQ ID NO: 9 - DNA, 69 bp, mus musculus
ctcaccctaactgtaaagtaattgtgtgttttgagactataaatatcccttggagaaaagccttgtttg

SEQ ID NO: 10 - DNA, 69 bp, Artificial
  Synthetic construct
ctcaccctaactgtaaagtaattataacttcgtatagtataaattatacgaagttataagccttgtttg

SEQ ID NO: 11 - DNA, 1781 bp, Artificial
  Synthetic construct
gatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattataacttcgtatagtataaattatacgaagttataagccttggttttttgaattccgtattaccgccatgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtagcggccgcataacttcgtatagtataaattatacgaagttataagccttgtttgagctgtttctgaggagccttcaagagaggctcctcagaaacagctcttttttc

SEQ ID NO: 12 - DNA, 55 bp, Artificial; identical to SEQ ID NO: 5
  Synthetic construct

SEQ ID NO: 13 - DNA, 68 bp, lentivirus
aaaaaaccaaggcttataacttcgtataatttatactatacgaagttataattactttacagttaccc

SEQ ID NO: 14 - DNA, 55 bp, mus musculus
tgtactctcctcccctcaatttcaagagaattgaggggaggagagtacttttttc

SEQ ID NO: 15 - DNA, 59 bp, mus musculus
tcgagaaaaaagtactctcctcccctcaattctcttgaaattgaggggaggagagtaca

SEQ ID NO: 16 - DNA, 26 bp, lentivirus
cccggttaatttgcatataatatttc

SEQ ID NO: 17 - DNA, 24 bp, lentivirus
catgatacaaaggcattaaagcag

SEQ ID NO: 18 - DNA, 55 bp, mus musculus
tggctgacaaagactatcacttcaagagagtgatagtctttgtcagccttttttc

SEQ ID NO: 19 - DNA, 59 bp, mus musculus
tcgagaaaaaaggctgacaaagactatcactctcttgaagtgatagtctttgtcagcca

SEQ ID NO: 20 - DNA, 53 bp, mus musculus
tgagtgtgtgagggagaaattcaagagatttctccctcacacactcttttttc

SEQ ID NO: 21 - DNA, 57 bp, mus musculus
tcgagaaaaaagagtgtgtgagggagaaatctcttgaatttctccctcacacactca

SEQ ID NO: 22 - DNA, 23 bp, lentivirus
tcgagtactaggatccattaggc

SEQ ID NO: 23 - DNA, 23 bp, lentivirus
ggccgcctaatggatcctagtac

SEQ ID NO: 24 - DNA, 19 bp, Mus musculus
gtactctcctcccctcaat

(19) United States
(12) Patent Application Publication
(10) Pub. No.: US 2005/0289659 A1
(43) Pub. Date: Dec. 29, 2005

(54) Cre-lox based method for conditional RNA interference

(76) Inventors: E. Tyler Jacks, West Newton, MA (US); Andrea Ventura, Cambridge, MA (US)

Correspondence Address: PEARL COHEN ZEDEK, LLP, 10 ROCKEFELLER PLAZA, SUITE 1001, NEW YORK, NY, 10020, US

(21) Appl. No.: 11/108,890

(22) Filed: Apr. 19, 2005

Related U.S. Application Data
(60) Provisional application No. 60/571,888, filed on May 18, 2004.

(51) Int. Cl. A01K 67/027

(52) U.S. Cl. 800/14; 800/21

(57) ABSTRACT

The present invention relates to vectors, compositions and methods for conditional, Cre-lox regulated, RNA interference. Vectors for use in conditional expression of a coding sequence based on a strategy in which the mouse U6 promoter is modified to include a hybrid between a LoxP site and a TATA box, and their use in conditional expression in transgenic mice are disclosed. The vectors allow for spatial and temporal control of miRNA expression in vivo.

### FIELD OF THE INVENTION

The present invention relates to vectors and their use in a cre-lox based method for conditional RNA interference.

### BACKGROUND OF THE INVENTION

RNA interference (RNAi) has emerged as a powerful tool to silence gene expression, and has rapidly transformed gene function studies across phyla. RNAi operates through an evolutionarily conserved pathway that is initiated by double-stranded RNA (dsRNA). In model eukaryotes such as plants and worms, long dsRNA (eg. 1000 nt) introduced into cells is processed by the dsRNA ribonuclease Dicer into ˜21 nt small-interfering RNAs (siRNAs). siRNAs in turn associate with an RNAi-induced silencing complex (RISC) and direct the destruction of mRNA complementary to one strand of the siRNA. Although the Dicer pathway is highly conserved, introduction of long dsRNA (>30 nt) into mammalian cells results in the activation of antiviral pathways leading to non-specific inhibition of translation and cytotoxic responses. The use of synthetic siRNAs that transiently down-modulate target genes, is one way to circumvent the cytotoxic dsRNA-activated pathways in mammals.

An important advance in the RNAi field was the discovery that plasmid-based RNA interference can substitute for synthetic siRNAs, thus permitting the stable silencing of gene expression. In such systems an RNA polymerase III promoter is used to transcribe a short stretch of inverted DNA sequence, which results in the production of a short hairpin RNA (shRNA) that is processed by Dicer to generate siRNAs. These vectors have been widely used to inhibit gene expression in mammalian cell systems.

More recently, several groups have reported the use of RNA polymerase III-based shRNA expression constructs to generate transgenic RNAi mice, in some cases recapitulating knock-out phenotypes. Due to the dominant nature of RNAi, a major limitation of this approach is that germ-line transmission can be obtained only for shRNAs targeting genes whose knock-down is compatible with animal viability and fertility. Moreover, even for cell-based applications, constitutive knock-down of gene expression by RNAi can limit the scope of experiments, especially for genes whose inhibition leads to cell lethality.

Thus, there is a great need for a widely applicable means of conditional knockdown of gene expression, without these limitations, and in particular, a need for rapid and cost effective generation of conditional “knockdown” animals and cell lines on a scale suitable for functional genomic studies.

### SUMMARY OF INVENTION

The present invention discloses, in one embodiment, a method of conditionally reducing expression of a coding sequence in a target cell, said method comprising contacting said target cell with a vector comprising:
i. An RNA Polymerase III promoter engineered to comprise a TATA-lox sequence;
ii. A transcriptional terminator sequence downstream of said TATA-lox sequence; and
iii. A second TATA-lox sequence upstream of an miRNA agent specific for said coding sequence, wherein said second TATA-lox sequence is downstream of said transcriptional terminator sequence;
   wherein said target cell is capable of expressing a Cre recombinase and whereby, following Cre-mediated recombination, said miRNA agent is expressed and reduces expression of said coding sequence, thereby conditionally reducing expression of a coding sequence in a target cell.

In another embodiment, this invention provides a method of conditionally expressing a coding sequence in a target cell, the method comprising contacting said target cell with a vector comprising:
i. An RNA Polymerase III promoter immediately downstream of a loxP site;
ii. An miRNA agent specific for said coding sequence, operatively-linked thereto; and
iii. A loxP site downstream of said miRNA agent;
   wherein said cell expresses said miRNA agent, thereby reducing expression of said coding sequence and whereby, following expression of said miRNA agent, Cre-mediated recombination is enabled in said target cell, such that said miRNA agent is no longer expressed, thereby being a method of conditionally expressing a coding sequence in a target cell.

In another embodiment, this invention provides a vector comprising:
i. An RNA Polymerase III promoter engineered to comprise a TATA-lox sequence;
ii. A transcriptional terminator sequence downstream of said TATA-lox sequence; and
iii. A second TATA-lox sequence upstream of an miRNA agent specific for said coding sequence, wherein said second TATA-lox sequence is downstream of said transcriptional terminator sequence;

In another embodiment, this invention provides a method of producing an animal genetically inactivated for a coding sequence, the method comprising:
a. contacting an embryonic stem cell with a vector of this invention;
b. injecting the embryonic stem cell in (a) to a blastocyst of said animal; and
c. obtaining an animal in (b) expressing said vector
   whereby, following Cre-mediated recombination in said animal, said miRNA agent is expressed and reduces expression of said coding sequence, thereby being a method of producing an animal genetically inactivated for a coding sequence.

In another embodiment, this invention provides a method of producing an animal genetically inactivated for a coding sequence, the method comprising:
a. contacting a single cell embryo of said animal with a vector of this invention; and
b. obtaining an animal expressing said vector
   whereby, following Cre-mediated recombination in said animal, said miRNA agent is expressed and reduces expression of said coding sequence, thereby being a method of producing an animal genetically inactivated for a coding sequence.

In another embodiment, this invention provides a method of identifying a gene product involved in carcinogenesis, the method comprising:
a. Obtaining an animal of this invention, wherein said coding sequence is for a gene product which is putatively involved in carcinogenesis;
b. Maintaining the animal in (a) under conditions facilitating carinogenesis;
c. Initiating or enabling Cre-mediated recombination in the animal in (b); and
d. Identifying the inhibition or suppression of carcinogenesis in the animal in (c),
   wherein inhibition or suppression of carcinogenesis in said animal indicates said coding sequence is from a gene whose product is involved in carcinogenesis.

In another embodiment, this invention provides a vector comprising:
i. An RNA Polymerase III promoter immediately downstream of a loxP site;
ii. An miRNA agent specific for said coding sequence, operatively-linked thereto; and
iii. A loxP site downstream of said miRNA agent;

In another embodiment, this invention provides a method of producing an animal genetically reactivated for a coding sequence, the method comprising:
a. contacting an embryonic stem cell with a vector comprising:
   1. an RNA Polymerase III promoter immediately downstream of a loxP site;
   2. An miRNA agent specific for said coding sequence, operatively -linked thereto; and
   3. A loxP site downstream of said miRNA agent;
b. injecting the embryonic stem cell in (a) to a blastocyst of said animal; and
c. obtaining an animal in (b) expressing said vector
   whereby, following Cre-mediated recombination in said animal, said miRNA agent is no longer expressed and said coding sequence is expressed, thereby being a method of producing an animal genetically reactivated for a coding sequence.

In another embodiment, this invention provides a method of producing an animal genetically reactivated for a coding sequence, the method comprising:
a. contacting a single cell embryo of said animal with a vector comprising:
   1. an RNA Polymerase III promoter immediately downstream of a loxP site;
   2. An miRNA agent specific for said coding sequence, operatively -linked thereto; and
   3. A loxP site downstream of said miRNA agent; and
b. obtaining an animal expressing said vector
   whereby, following Cre-mediated recombination in said animal, said miRNA agent is no longer expressed and said coding sequence is expressed, thereby being a method of producing an animal genetically reactivated for a coding sequence.

In another embodiment, this invention provides a method of identifying a tumor suppressor gene, the method comprising:
a. Obtaining an animal this invention, which is genetically reactivated for a coding sequence, wherein said coding sequence is for a putative tumor suppressor;
b. Maintaining the animal in (a) under conditions promoting carcinogenesis;
c. Initiating or enabling Cre-mediated recombination the animal in (b) following carcinogenesis; and
d. Identifying inhibition or suppression of carcinogenesis in the animal in (c);
   wherein inhibition or suppression of carcinogenesis in said animal indicates said coding sequence is from a tumor suppressor gene.

### BRIEF DESCRIPTION OF THE DRAWINGS

FIG. 1 depicts the organization and expression of constructs comprising a TATAlox promoter. A. Shematic representation of the mouse U6 promoter drawn to scale The spacing between the distal sequence element (DSE), the proximal sequence element (PSE), the TATA box and the transcription start site (+1) are indicated. B. Comparison between the sequence of a loxP site and a TATAlox site (upper panel) and (lower panel) comparison between the sequence of the wild type mouse U6 promoter and the sequence of the U6 promoter with a TATAlox site replacing the TATA box C. Equal amounts of the wt U6 promoter and of the TATA lox U6 promoter (empty or driving the expression of oligos against the firefly luciferase gene) were transfected in 293T cells together with reporter plasmids expressing firefly luciferase and renilla luciferase. 36 hours later cells were lysed and the ratio between firefly and renilla luciferase activity was measured. D. Increasing amounts (0 to 200 ng) of a plasmid containing either the 2 TATA lox U6 promoter-luciferase shRNA or of the 1 TATA lox U6-luciferase shRNA were transfected in 293T cells together with reporter plasmids expressing firefly luciferase and renilla luciferase and luciferase activity was measured as in FIG. 1C. E. Schematic representation of pSico before and after Cre mediated recombination. F. Schematic representation of pSicoR before and after Cre mediated recombination.

FIG. 2l is a schematic representation of the U6 promoters carrying the lox-CMVGFP-lox tested in panel 1B. The CMV-GFP cassette is not drawn to scale. Test1 and Test2 have the lox-STOP-lox cassette between the DSE and the PSE. In Test3 the cassette is positioned between the PSE and the TATA box and finally in Test4 it is positioned between the TATAbox and the putative transcription start site. B. The indicated U6 constructs were assayed as in FIG. 1C for their ability to induce knock-down of the firefly luciferase gene. Note that constructs containing the lox-stop-lox cassette upstream of the PSE are still capable of efficiently repressing luciferase activity (Test 1 and Test 2), while the constructs in which the lox-stop-lox cassette is situated between the PSE and the TATA (Test 3) or between the TATA and the transcription start site (Test 4), are inactive even in the recombined conformation indicating that in both cases the residual lox site negatively affects U6 promoter activity

FIG. 3 demonstrates expression and knockdown ability of lentiviral pSico vectors in the presence or absence of Cre. A. p53^{R270H/-} MEF infected with the indicated lentiviruses were sorted for GFP positivity and infected with Adeno empty or Adeno Cre. Four days after infection genomic DNA was extracted and a PCR reaction was performed to amplify the recombined and unrecombined viral DNA. B. The same cells were analysed by epifluorescence microscopy to detect GFP fluorescence. Similar cell density and identical exposure time was used for all images. C. 15 μg of total RNA extracted from the above indicated MEFs were separated on a 15% denaturing polyacrilamide gel, transferred on a nitrocellulose filter and hybridized to a radio-labeled 19mer corresponding to the sense strand of the p53 shRNA. Equal RNA loading was assessed by ethidium bromide staining of the upper part of the gel (lower panel) D. Northern blotting (upper panel) and western blotting (lower panel) showing p53 knockdown in the above indicated cells. E. Cell cycle profile of wild type MEFs infected with the indicated lentiviruses, superinfected with Adeno empty or AdenoCre and either mock treated or treated with 1 μg/ml doxorubicin for 12 hours (as indicated). The experiment on pSico Luc and pSico p53 was performed 4 days after Adeno infection, while the experiment on pSicoR p53 and control cells was performed 10 days after Adeno infection to allow (see text for details). F. Whole cell lysates from cells described in panel 1E were separated by PAGE and immunoblotted against p53 and tubulin.

FIG. 4 demonstrates knockdown and conditional expression of lentiviral pSico and pSico R vectors in the presence or absence of Cre. A. MEFs were infected with the indicated lentiviruses, GFP positive cells were sorted and superinfected with empty Adenovirus or AdenoCre. One week later whole cell lysates were separated by SDS-PAGE subjected to western blotting against Npm and tubulin. B. Embryonic stem cells carrying a doxycycline-inducible Cre (C. Beard and R. Jaenisch, unpublished data) were infected with the indicated lentiviruses. GFP positive clones were isolated, passaged two times and either left untreated or incubated with 10 μg/ml doxycycline for 1 week. Immunoblot analysis was performed as in panel A. C. Immunofluorescence microscopy analysis of MEFs infected with pSico-Npm, pSicoR-Npm or pSico-CD8. After lentiviral infection GFP-positive MEFs were sorted and superinfected with empty Adenovirus or Ad-Cre. One week later cells were co-plated on glass coverslips, fixed and decorated with anti Npm antibody (red). Nuclei were stained with DAPI. D. Methylation analysis of minor satellites DNA. ES cells carrying a doxycycline-inducible Cre transgene were infected with the indicated lentiviruses. Single GFP positive clones were isolated, expanded and passaged 5 times before being either mock treated or incubated with 2 μg/ml doxycycline. After five more passages the genomic DNA was extracted and digested with the indicated enzymes and subjected to Southern blot analysis. E. As in panel D, but the genomic DNA was treated with sodium bisulfite, subjected to PCR to amplify the indicated imprinted regions and digested with BstUI.

FIG. 5 demonstrates conditional RNAi in mice. A. ES cells infected with pSico-CD8 visualized with an inverted fluorescence microscope. B. A litter of newborns derived from a cross between a pSico-CD8 chimera and a Lck cre female. Two pups present bright GFP fluorescence indicating germ-line transmission of the pSico-CD8 transgene. C. Knockdown of CD8 in the spleen of Msx2-Cre x pSico-CD8 and Lck-Cre x pSico-CD8 mice. Chimeras from pSico-CD8-infected ES cells were crossed to Msx2-Cre or Lck-Cre animals. The resulting mice were genotyped for the presence of Cre and pSico. Splenocytes from 1-3 weeks old mice with the indicated genotypes were harvested, stained for CD3, CD4 and CD8 expression and analyzed by flow cytometry. Only CD3+ cells were plotted. One representative example of littermates for each cross is shown. D. PCR detection of pSico-CD8 Cre-mediated recombination in genomic DNA extracted from the tail (A) or the thymus (B) of mice with the indicated genotypes.

FIG. 6 demonstrates tetraploid blastocyst complementation with pSico-infected ES cells. A. A day 14.5 p.c. embryo derived by tetraploid complementation using the pSico-p53 #1 ES clone. The area enclosed by the dashed line corresponds to the non-ES cell-derived placenta. B. PCR detection of recombination in MEFs derived from the indicated embryos. Genomic DNA was extracted four days after Adeno or Adeno-Cre infection and subjected to PCR. C. Histogram overlays showing loss of GFP expression in MEFs derived from pSico-p53#1 (upper) and pSico-p53#3 (lower) embryos 4 days after Adeno Cre (green plot) or Adeno empty (purple filled plot) infection. Control, GFP negative MEFs (red plot) are included as reference. D. Cell cycle profile of MEFs derived from embryos with the indicated genotypes infected with Adeno empty or AdenoCre and either mock treated or treated with 1 μg/ml doxorubicin for 18 hours. E. As in panel D, but the cells were lysed and subjected to western blot against p53 and tubulin.

### DETAILED DESCRIPTION OF THE INVENTION

The present invention provides, in one embodiment, methods for conditionally reducing expression of a coding sequence in a cell or animal, comprising contacting the cell with a vector comprising a TATAlox-stop-lox cassette, upstream of an miRNA agent specific for the coding sequence, in cells capable of expressing a Cre recombinase.

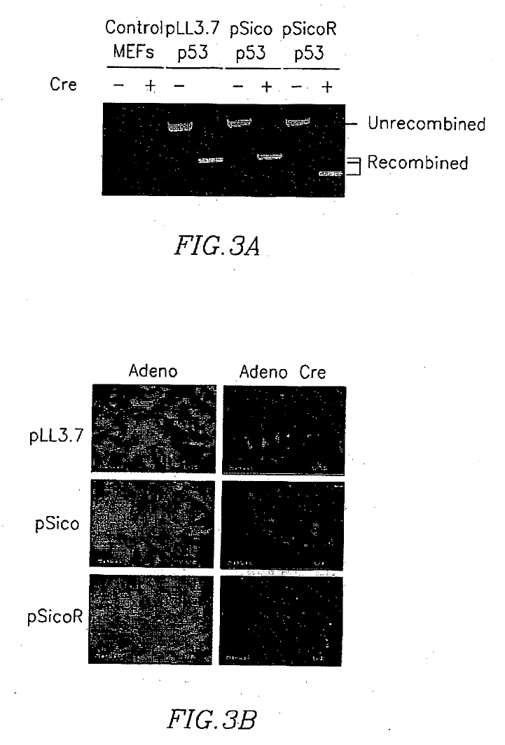

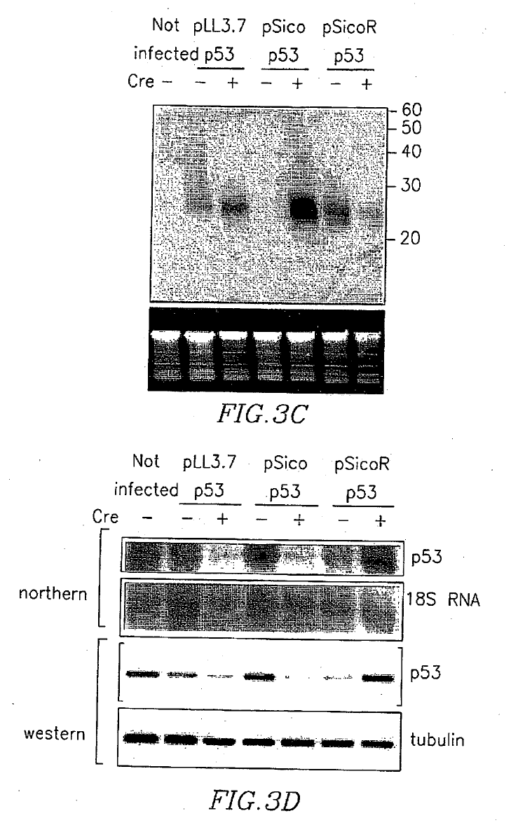

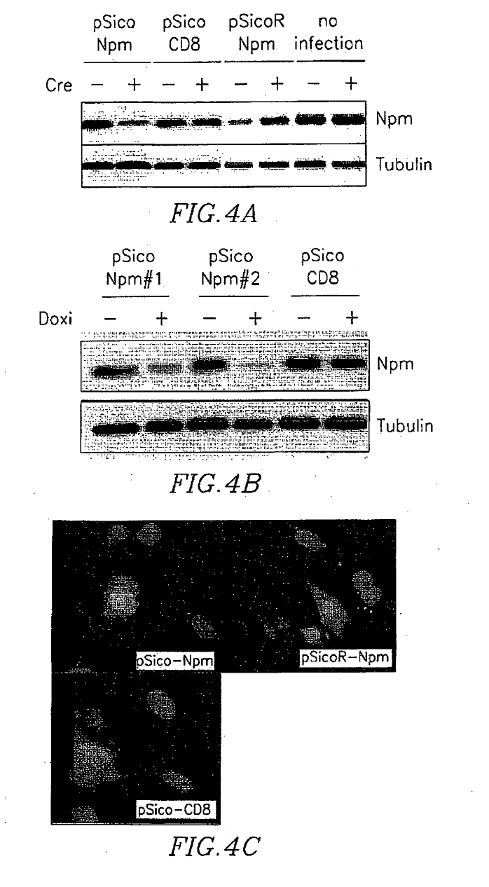

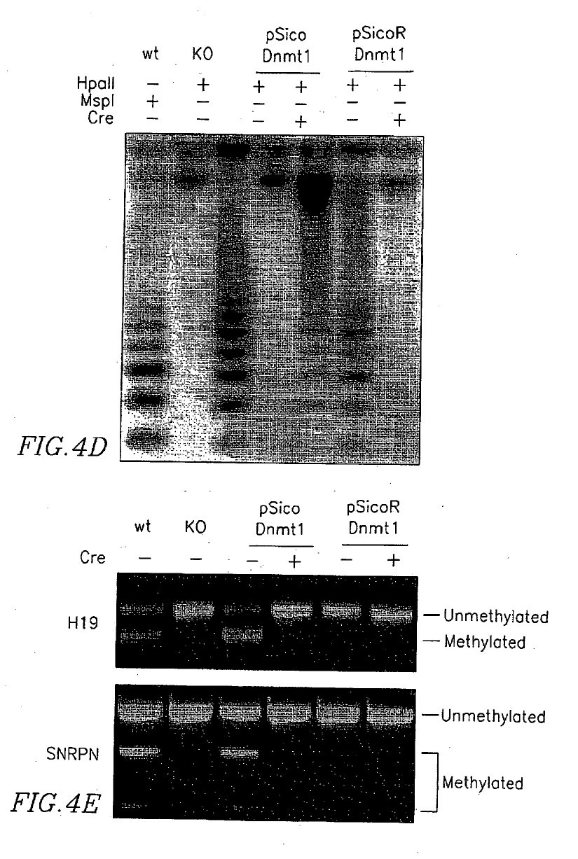

Conditionally reduced expression of a coding sequence was demonstrated herein, with the use of a lentiviral vector pSico, which comprises a modified U6 promoter comprising a TATAlox , a transcriptional terminator downstream of the TATAlox, and a reporter gene operatively linked to a second promoter downstream of the transcriptional terminator. The reporter gene was upstream of an additional TATAlox sequence, and the lox sequence was upstream of an shRNA, specific for p53. Cre expression in MEF cells infected with pSico expressed the shRNA, and demonstrated reduced p53 protein levels (FIG. 3). Similarly pSico lentiviral vectors comprising shRNA for NPM demonstrated silenced NPM and Dnmt1 gene expression in a Cre-dependent fashion (FIG. 4).

In one embodiment, this invention provides a method of conditionally reducing expression of a coding sequence in a target cell, said method comprising contacting said target cell with a vector comprising:
i. An RNA Polymerase III promoter engineered to comprise a TATA-lox sequence;
ii. A transcriptional terminator sequence downstream of said TATA-lox sequence; and
iii. A second TATA-lox sequence upstream of an miRNA agent specific for said coding sequence, wherein said second TATA-lox sequence is downstream of said transcriptional terminator sequence;
   wherein said target cell is capable of expressing a Cre recombinase and whereby, following Cre-mediated recombination, said miRNA agent is expressed and reduces expression of said coding sequence, thereby conditionally reducing expression of a coding sequence in a target cell.

In one embodiment, the term “vector” refers to a nucleic acid molecule capable of transporting another nucleic acid to which it has been linked. In one embodiment, the vector is a genomic integrated vector, or “integrated vector”, which can become integrated into the chromsomal DNA of the host cell. In another embodiment, the vector is an episomal vector, i.e., a nucleic acid capable of extra-chromosomal replication in an appropriate host, such as, for example a eukaryotic host cell. The vector according to this aspect of the present invention may be, in other embodiments, a plasmid, a bacmid, a phagemid, a cosmid, a phage, a virus or an artificial chromosome.

A nucleic acid of the present invention will generally contain phosphodiester bonds in one embodiment, or in another embodiment, nucleic acid analogs are included, that may have alternate backbones, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate, phosphorodithioate, O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). These modifications of the ribose-phosphate backbone or bases may be done to facilitate the addition of other moieties such as chemical constituents, including 2′O-methyl and 5′ modified substituents, or to increase the stability and half-life of such molecules in physiological environments.

The nucleic acids may be single stranded or double stranded, or contain portions of both double stranded or single stranded sequence. The nucleic acid may be DNA, both genomic and cDNA, RNA or a hybrid, where the nucleic acid contains any combination of deoxyribo-and ribo-nucleotides, and any combination of bases, including uracil, adenine, thymine, cytosine, guanine, inosine, xathanine and hypoxathanine, etc. Thus, for example, chimeric DNA-RNA molecules may be used such as described in Cole-Strauss et al., Science 273:1386 (1996) and Yoon et al., PNAS USA 93:2071 (1996).

In another embodiment, this invention provides a vector comprising an RNA Polymerase III promoter engineered to comprise a TATA-lox sequence, a transcriptional terminator sequence downstream of the TATA-lox sequence and a second TATA-lox sequence upstream of an miRNA agent specific for the coding sequence, wherein the second TATA-lox sequence is downstream of said transcriptional terminator sequence. In another embodiment, this invention provides a composition comprising the vector.

The vectors of this invention comprise, inter alia, an miRNA agent specific for a coding sequence.

The term “miRNA agent” refers, in one embodiment, to an agent that modulates expression of a target gene by an RNA interference mechanism. Micro-RNAs are a very large group of small RNAs produced naturally in organisms, which in one embodiment, regulates the expression of target genes. Founding members of the micro-RNA family are let-7 and lin-4. The let-7 gene encodes a small, highly conserved RNA species that regulates the expression of endogenous protein-coding genes during worm development. The active RNA species is transcribed initially as an ⁻70 nt precursor, which is post-transciptionally processed into a mature ⁻21 nt form. Both let-7 and lin4 are transcribed as hairpin RNA precursors, which are processed to their mature forms by Dicer enzyme.

In one embodiment the miRNA agent comprises double-stranded RNA, which can form a hairpin structure. The miRNA agents employed, in another embodiment, are small ribonucleic acid molecules, or oligoribonucleotides, that are present in duplex structures, such as, in one embodiment, two distinct oligoribonucleotides hybridized to each other, or in another embodiment, a single ribooligonucleotide that assumes a hairpin structure to produce a duplex structure.

In one embodiment, miRNA agent does not exceed about 100 nt in length, and typically does not exceed about 75 nt length, where the length in certain embodiments is less than about 70 nt. In one embodiment, the miRNA agent of this invention has a length about 15 to 40 bp, or in another embodiment, about 20 and 29 bps, or in another embodiment, 25 and 35 bps, or in another embodiment, about 20 and 35 bps, or in another embodiment, about 20 and 40 bps, or in another embodiment, 21 bp, or in another embodiment, 22 bp.

In one embodiment, the nucleic acids/oligonucleotides comprising the miRNA agent may be synthesized on an Applied Bio Systems oligonucleotide synthesizer according to specifications provided by the manufacturer In another embodiment, the nucleic acids/oligonucleotides or modified oligonucleotides may be synthesized by any number of means as is generally known in the art, and as is described hereinbelow.

In one embodiment, the miRNA agent encodes an interfering ribonucleic acid. In one embodiment, the miRNA agent is a transcriptional template of the interfering ribonucleic acid. According to this aspect of the invention, and in one embodiment, the transcriptional template is typically a DNA that encodes the interfering ribonucleic acid. The DNA may be present in a vector, such as, and in one embodiment, a plasmid vector, or in another embodiment, a viral vector, or any other vector, as will be known to one skilled int the art.

In one embodiment, the term “coding sequence” refers to a nucleic acid sequence that “encodes” a particular polypeptide or peptide. In one embodiment, the coding sequence is a nucleic acid sequence that is transcribed (in the case of DNA) and is translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from procaryotic or eukaryotic mRNA, genomic DNA sequences from procaryotic or eukaryotic DNA, and even synthetic DNA sequences. A transcription termination sequence will usually be located 3′ to the coding sequence.

In one embodiment the term “coding sequence”, includes DNA sequences that encode a polypeptide, as well as DNA sequences that are transcribed into inhibitory antisense molecules.

In one embodiment, the term “reducing expression”, as it refers to vectors and their use according to the methods of this invention, refers to a diminishment in the level of expression of a gene when compared to the level in the absence of the miRNA agent.

In one embodiment, reduced expression may be affected at the transcriptional or translational level, or a combination thereof.

According to this aspect of the invention, reduced expression using the vectors, and/or according to the methods of this invention, is specific. In one embodiment, the reduction in expression is via an ability to inhibit a target gene without manifest effects on other genes of the cell. The consequences of inhibition can be confirmed, in other embodiments, by examination of the outward properties of the cell or organism or by biochemical techniques such as RNA solution hybridization, nuclease protection, Northern hybridization, gene expression monitoring with a microarray, antibody binding, enzyme linked immunosorbent assay (ELISA), Western blotting, radioimmunoassay (RIA), other immunoassays, and fluorescence activated cell analysis (FACS).

In one embodiment, the miRNA agent is an shRNA, which specifically inactivates p53, nucleolar protein nucleophosmin (NPM) or DNA methyltransferase (DNMT-1) gene expression, as exemplified hereinbelow.

In one embodiment, the vectors and methods of utilizing the same for reducing expression of a target gene may result in inhibition of target gene expression of greater than 10%, 33%, 50%, 75%, 80%, 85%, 90%, 95% or 99% as compared to a cell not subjected to the vectors and methods of utilizing the same for reducing expression. In another embodiment, lower doses of administered miRNA agent, and longer times following administration may result in inhibition in a smaller fraction of cells (e.g., at least 10%, 20%, 50%, 75%, 90%, or 95% of targeted cells)

In one embodiment, this invention provides for a method of conditionally reduced expression of a coding sequence in a target cell. In one embodiment, the term “conditionally reduced expression” refers to the flexibility inherent in the methods/vectors of this invention, which enable regulation of reducing expression of a coding sequence in a target cell. In one embodiment, reducing expression via the vectors/methods of this invention is controlled over time, or in a cell or tissue-specific manner, such that production of the miRNA agent is not constant.

Expression of the miRNA agent within a target cell, in one embodiment of this invention, takes advantage of a lox/cre system. In one embodiment, miRNA agent expression is dependent upon the presence of a Cre recombinase.

In one embodiment, the cre recombinase, is derived from a P1 bacteriophage (Abremski and Hoess, J. Biol. Chem. 259(3):1509-1514 (1984)) which acts on a specific 34 base pair DNA sequence known as “loxP” (locus of crossover), which is, in turn, comprised of two 13 base pair inverted repeats (serving as the recombinase binding sites) flanking an 8 base pair core sequence (Current Opinion in Biotechnology 5:521-527 (1994). Cre catalyzes the rearrangement of DNA sequences that contain loxP sites Recombination between two loxP sites (catalyzed by the cre protein) causes, in certain cases, the loss of sequences flanked by these sites [for a review see N. Kilby et al, Trends Genet., 9:413-421 (1993)].

According to this aspect of the invention, and in one embodiment, the mutant lox site containing a functional TATA box in its spacer sequence enabled cre-regulated transcription and efficient processing of a normal-length shRNA (FIG. 3C). The amount of processed shRNA was even higher in cells containing the TATAlox U6 promoter, as compared to cells containing the wild-type promoter (FIG. 3C). In one embodiment, this is a result of enhanced transcriptional activity.

In one embodiment of this invention, the promoter comprises the first loxP sequence, with the miRNA agent linked to the second loxP sequence, where miRNA expression arising as a result of the site-specific recombination, mediated by cre. According to this aspect of the invention, cre-dependent miRNA agent expression is initiated following site-specific recombination, and in one embodiment, is in a location-controlled or, in another embodiment, time-controlled manner, or in another embodiment, is controlled by a combination thereof

Cre works in simple buffers, such as, in one embodiment, with magnesium or, in another embodiment, spermidine as a cofactor, as is well known in the art. The DNA substrates acted on by cre may be, in one embodiment, in linear, or, in another embodiment, in a supercoiled configuration.

In one embodiment, the cre sequence is as that described in N. Sternberg et al, J. Mol. Biol., 187:197-212 (1986). In another embodiment, the cre recombinase may be obtained from commercial sources (for example from Novagen, Catalog No. 69247-1).

In one embodiment, cre recombinase will be expressed in a target cell of this invention. In another embodiment, the target cell will be engineered to express cre by any means as will be known to one skilled in the art.

The vectors and methods utilizing the same, of this invention, make use of a lox/cre system, where, in one embodiment, canonical lox P sites are utilized. According to this aspect of the invention, in one embodiment, the loxP site may have a nucleic acid sequence corresponding to or homologous to ATAACTTCGTATAGCATACATTATACGAAGTTAT (SEQ ID NO: 8).

In one embodiment, the terms “homology”, “homologue” or “homologous”, refer to a, which exhibits, in one embodiment at least 70% correspondence with the indicated sequence. In another embodiment, the sequence exhibits at least 72% correspondence with the indicated sequence. In another embodiment, the sequence exhibits at least 75% correspondence with the indicated sequence. In another embodiment, the sequence exhibits at least 80% correspondence with the indicated sequence. In another embodiment, the sequence exhibits at least 82% correspondence with the indicated sequence. In another embodiment, the sequence exhibits at least 85% correspondence with the indicated sequence. In another embodiment, the sequence exhibits at least 87% correspondence with the indicated sequence. In another embodiment, the sequence exhibits at least 90% correspondence with the indicated sequence. In another embodiment, the sequence exhibits at least 92% correspondence with the indicated sequence. In another embodiment, the sequence exhibits at least 95% or more correspondence with the indicated sequence. In another embodiment, the sequence exhibits at least 97% correspondence with the indicated sequence. In another embodiment, the sequence exhibits at least 99% correspondence with the indicated sequence. In another embodiment, the sequence exhibits 95% -100% correspondence with the indicated sequence. Similarly, as used herein, the reference to a correspondence to a particular sequence includes both direct correspondence, as well as homology to that sequence as herein defined.

Homology, as used herein, may refer to sequence identity, or may refer to structural identity, or functional identity. By using the term “homology” and other like forms, it is to be understood that any molecule, that functions similarly, and/or contains sequence identity, and/or is conserved structurally so that it approximates the reference sequence, is to be considered as part of this invention.

Homology may be determined in the latter case by computer algorithm for sequence alignment, by methods well described in the art. For example, computer algorithm analysis of nucleic acid sequence homology may include the utilization of any number of software packages available, such as, for example, the BLAST, DOMAIN, BEAUTY (BLAST Enhanced Alignment Utility), GENPEPT and TREMBL packages.

An additional means of determining homology is via determination of candidate sequence hybridization, methods of which are well described in the art (See, for example, Nucleic Acid Hybridization, Hames and Higgins, Eds. (1985); Molecular Cloning, Sambrook and Russell, eds. (2001), and Current Protocols in Molecular Biology, Ausubel et al. eds, 1989). For example, methods of hybridization may be, in one embodiment, carried out under moderate to stringent conditions, to the complement of a DNA encoding a native peptide or protein of interest. Hybridization conditions may be, for example, overnight incubation at 42° C. in a solution comprising: 10-20% formamide, 5×SSC (150 millimolar (mM) NaCl, 15 mM trisodium citrate), 50 mM sodium phosphate (pH 7.6), 5× Denhardt's solution, 10% dextran sulfate, and 20 micrograms (μg)/milliliter (ml) denatured, sheared salmon sperm DNA. Each method represents a separate embodiment of the present invention.

In another embodiment, mutated loxP sites, may be employed in the vectors and/or methods of this invention. In one embodiment, the mutated lox P site will comprise a TATAlox sequence, such as that exemplified further hereinunder.

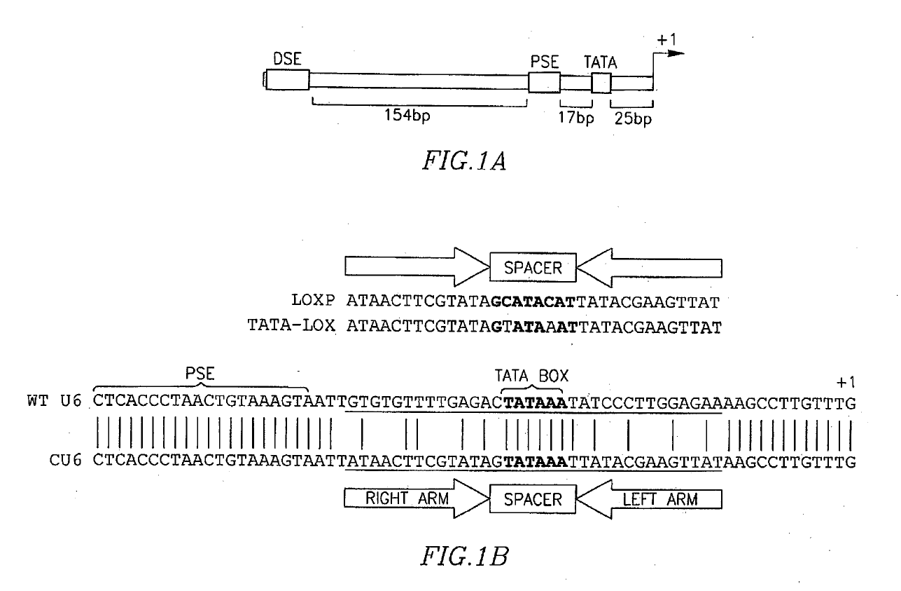

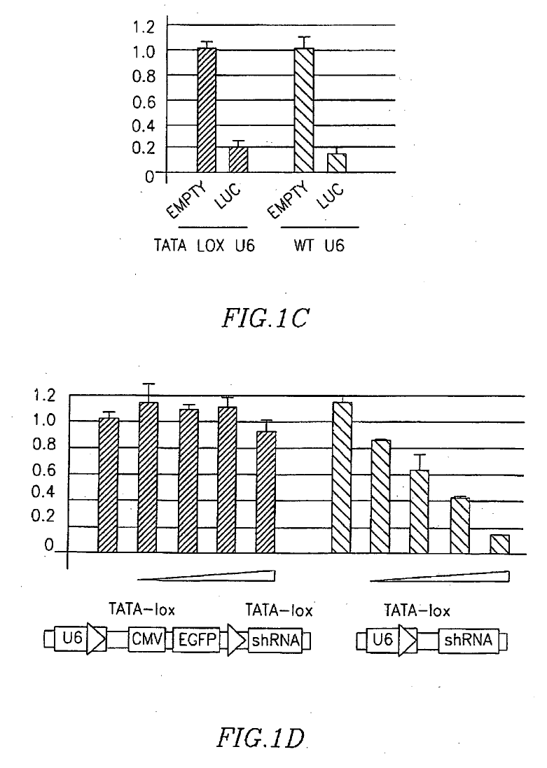

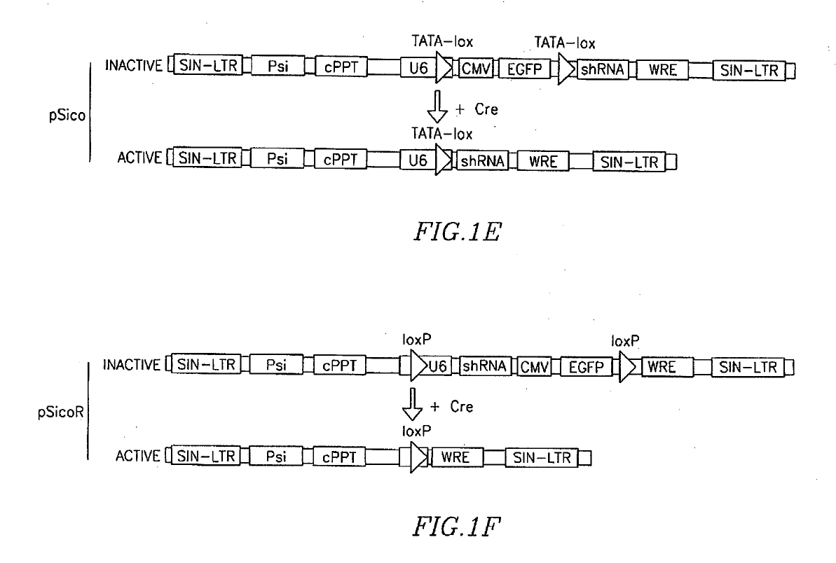

In one embodiment, the term TATAlox refers to a bifunctional lox site that is capable of undergoing Cre-mediated recombination, and contains a functional TATA box. In one embodiment, the TATA box is in the spacer region (FIG. 1, C). In one embodiment, the TATAlox will comprise a nucleotide sequence corresponding to or homologous to:

| | |
|---|---|
| ATAACTTCGTATAGTATAAATTATACGAAGTTAT. | (SEQ ID NO: 7) |

According to this aspect of the invention, and in one embodiment, the vectors of this invention comprise a promoter, which comprises the TATAlox sequence.

In one embodiment, the term “promoter” refers to a nucleic acid sequence, which regulates expression of a nucleic acid, operably linked thereto. Such promoters are known to be cis-acting sequence elements required for transcription as they serve to bind DNA dependent RNA polymerase, which transcribes sequences present downstream thereof.

The term “operably linked”, in one embodiment, refers to a relationship permitting the sequences to function in their intended manner. A vector comprising a regulatory sequence “operably linked” to a coding sequence is ligated in such a way that expression of the nucleic acid sequence is achieved under conditions compatible with the control sequences.

In one embodiment, the promoter will be an RNA polymerase III promoter.

In one embodiment, the RNA polymerase III promoter will be a U6 or H1 promoter. According to this aspect of the invention, and as exemplified hereinbelow, the U6 promoter may be modified to incorporate TATA-lox, and has a nucleotide sequence corresponding to:

(SEQ ID NO: 10)

CTCACCCTAACTGTAAAGTAATTATAACTTCGTATAGTATAAATTATACG
AAGTTATAAGCCTTGTTTG.

In another embodiment, any promoter may be engineered to comprise a TATA-lox sequence.

In one embodiment, a promoter, including an engineered promoter used in the vectors and methods of this invention, may be one known to confer cell-type specific expression of a sequence operatively linked to thereto. For example, and in one embodiment, a promoter specific for myoblast gene expression can be operatively linked to an miRNA for a coding sequence of interest, a reporter gene, or a coding sequence of interest, to confer muscle-specific expression thereof. Muscle-specific regulatory elements which are known in the art include upstream regions from the dystrophin gene (Kiamut et al., (1989) Mol. Cell Biol.9:2396), the creatine kinase gene (Buskin and Hauschka, (1989) Mol. Cell Biol. 9:2627) and the troponin gene (Mar and Ordahl, (1988) Proc. Natl. Acad. Sci. USA. 85:6404).

In another embodiment, promoters used in the vectors and methods of this invention, specific for other cell types known in the art (e.g., the albumin enhancer for liver-specific expression; insulin regulatory elements for pancreatic islet cell-specific expression; various neural cell-specific regulatory elements, including neural dystrophin, neural enolase and A4 amyloid promoters) may be used, and represent an embodiment of this invention. In another embodiment, a promoter or regulatory element, which can direct constitutive expression of a sequence operatively linked thereto, in a variety of different cell types, such as a viral regulatory element, may be used. Examples of viral promoters commonly used to drive gene expression include those derived from polyoma virus, Adenovirus 2, cytomegalovirus and Simian Virus 40, and retroviral LTRs.

In another embodiment, a regulatory element, which provides inducible expression of a gene linked thereto, may be used. The use of an inducible promoter may allow, in another embodiment, for an additional means of modulating the product of the coding sequence in the cell. Examples of potentially useful inducible regulatory systems for use in eukaryotic cells include hormone-regulated elements (e.g., see Mader, S. and White, J. H. (1993) Proc. Natl. Acad. Sci. USA 90:5603-5607), synthetic ligand-regulated elements (see, e.g., Spencer, D. M. et al 1993) Science 262:1019-1024) and ionizing radiation-regulated elements (e.g., see Manome, Y. Et al. (1993) Biochemistry 32:10607-10613; Datta, R. et al. (1992) Proc. Natl. Acad. Sci. USA 89: 1014-10153). Additional tissue-specific or inducible regulatory systems may be developed for use in accordance with the invention.

According to this aspect of the invention, and in one embodiment, the vectors of this invention will comprise a promoter engineered to comprise a TATA-lox sequence, upstream of a transcriptional terminator sequence, which is upstream of a second TATA-lox sequence. In one embodiment, this arrangement is referred to as a TATAlox-stop-TATAlox cassette.

In one embodiment, the TATAlox-stop-TATAlox cassette is upstream of an miRNA agent, as described and exemplified herein, specific for a coding sequence.

In another embodiment, the vectors of this invention, comprising the TATAlox-stop-TATAlox cassette upstream of an miRNA agent are introduced into a target cell capable of expressing a Cre recombinase.

In one embodiment, the term “capable of expressing a Cre recombinase” refers to a cell that endogenously expresses the Cre recombinase, or in another embodiment, is engineered to express a Cre recombinase.

In one embodiment, the cell is in a culture system, or in another embodiment, in a body of a subject, or in another embodiment, is ex-vivo cultured, and following transfection or tranduction with a vector of this invention, is reintroduced to the subject from which the cell was taken. In one embodiment, the cell is a stem or progenitor cell. In another embodiment, the cell is a mature, differentiated cell. In one embodiment, the cell is a human cell in origin, or in another embodiment, the cell is murine in origin.

In one embodiment, the terms “Cells,” “host cells” or “target cells” are used interchangeably, and refer, in one embodiment, not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

In another embodiment, the cell is a diseased cell. In one embodiment, the cell is infected, or in another embodiment, the cell is transformed or neoplastic. In another embodiment, the cell is obtained from a subject with a disease whose etiology is associated with a genetic mutation. In another embodiment, the cell is obtained from a subject with a disease, where an inappropriate immune or inflammatory response has been initiated.

In one embodiment, the target cell of any method of the present invention may be a cancer cell or neoplastic cell. “Neoplastic cell” refers, in one embodiment, to a cell whose normal growth control mechanisms are disrupted (typically by accumulated genetic mutations), thereby providing potential for uncontrolled proliferation. Thus, “neoplastic cell” can include, in one embodiment, both dividing and non-dividing cells. In one embodiment, neoplastic cells may include cells of tumors, neoplasms, carcinomas, sarcomas, leukemias, lymphomas, and others. In another embodiment, “neoplastic cells” may include central nervous system tumors, such as, for example brain tumors. These may include, in other embodiments, glioblastomas, astrocytomas, oligodendrogliomas, meningiomas, neurofibromas, ependymomas, schwannomas or neurofibrosarcomas. In another embodiment, “neoplastic cells” can include either benign or malignant neoplastic cells. In another embodiment, “neoplastic cells” can include any other type of cancer known in the art.

In one embodiment, the target cell may be an infected cell. In another embodiment, the target cell may be a pathogenic cell. In another embodiment, the target cell may mediate autoimmunity or another disease state. In another embodiment, the target cell may comprise a mutated cellular gene necessary for a physiological function. In one embodiment, the mutated product results in disease in the subject. According to this aspect of the invention, the vectors/methods of this invention may be employed to silence a defective gene, and may futher be followed by delivery of a wild-type copy of the desired gene.

It is to be understood that any cell comprising a vector of this invention, or utilized for the methods of this invention, is to be considered as part of this invention, and represents an embodiment thereof.

According to this aspect of the invention, and in one embodiment, following Cre-mediated recombination in the target cell, the miRNA agent is expressed and reduces expression of the coding sequence, thereby conditionally reducing expression of a coding sequence in the target cell.

In another embodiment, the vector is a lentiviral vector. In one embodiment, the lentiviral vector of this invention may correspond to one as exemplified herein.

A lentiviral or lentivirus vector, as used herein, is a vector, which comprises at least one component part derivable from a lentivirus. In one embodiment, the component part is involved in the biological mechanisms by which the vector infects cells, expresses genes or is replicated. The term “derivable”, in one embodiment, refers to the fact that the sequence need not necessarily be obtained from a lentivirus but instead could be derived therefrom. By way of example, the sequence may be prepared synthetically or by use of recombinant DNA techniques.

The lentiviral vectors of this invention may be derived from any member of the family of lentiviridae. In one embodiment, the lentivirus may be a human immunodeificiency virus (HIV), a simian immunodeficiency virus (SIV), a feline immunodeficiency virus (FIV), a bovine immunodeficiency virus (BIV), a caprine arthritis encephalitis virus (CAEV), a Maedi/Visna virus (MVV) or an equine infectious anaemia virus (EIAV).

In one embodiment, the lentiviral vectors of this invention comprise sufficient lentiviral genetic information to allow packaging of an RNA genome, in the presence of packaging components, into a viral particle capable of infecting a target cell. In one embodiment, infection of the target cell includes reverse transcription and integration into the target cell genome. The lentiviral vectors of this invention may carry, in one embodiment, non-viral coding sequences which are to be delivered by the vector to the target cell. In one embodiment, the lentiviral vectors of this invention are incapable of independent replication to produce infectious retroviral particles within the final target cell. In one embodiment, the lentiviral vectors of this invention will lack a functional gag-pol and/or env gene and/or other genes essential for replication.

In one embodiment, the lentiviral vectors of this invention may be pseudotyped with any molecule of choice, including but not limited to envelope glycoproteins (wild type or engineered variants or chimeras) of VSV-G, rabies, Mokola, MuLV, LCMV, Sendai, or Ebola.

In one embodiment, the vectors of this invention may comprise other viral expression vectors. Viral vectors according to these aspects include but are not limited to other retroviral vectors, an adenoviral vector, an adeno-associated viral vector, a herpes viral vector, a pox viral vector, a parvoviral vector or a baculoviral vector.

In one embodiment, the retroviral vector employed in the present invention may be derived from or may be derivable from any suitable retrovirus, such as, for example, murine leukemia virus (MLV), human immunodeficiency virus (HIV), human T-cell leukemia virus (HTLV), mouse mammary tumour virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29) or Avian erythroblastosis virus (AEV), or others [see Coffin et al., 1997, “retroviruses”, Cold Spring Harbour Laboratory Press Eds: J M Coffin, S M Hughes, H E Varmus pp 758-763] each of which represents an embodiment of this invention.

In one embodiment, the vectors and methods of this invention may employ the use of enhancer sequences. In one embodiment, the term “enhancer” refers to a DNA sequence, which binds to other protein components of the transcription initiation complex and may thus facilitate the initiation of transcription directed by its associated promoter.

In another embodiment, the vectors and their use according to the present invention may further include a selectable marker. In one embodiment, the selectable marker comprises an antibiotic resistance cassette, by means well known to one skilled in the art. In one embodiment, the resistence cassette is for conferring resistance to ampicillin, bleomycin, chloramphenicol, gentamycin, hygromycin, kanamycin, lincomycin, methotrexate, phosphinothricin, puromycin, or tetracycline, or derivatives thereof.

In another embodiment, the selectable marker may comprise nucleic acid sequences encoding for a reporter protein, such as, for example, green fluorescent protein (GFP), DS-Red (red fluorescent protein), acetohydroxyacid synthase (AHAS), beta glucoronidase (GUS), secreted alkaline phosphatase (SEAP), beta-galactosidase, chloramphenicol acetyltransferase (CAT), horseradish peroxidase (HRP), luciferase, nopaline synthase (NOS), octopine synthase (OCS), or derivatives thereof, or any number of other reporter proteins known to one skilled in the art.

In another embodiment, the vector may further include an origin of replication, and may be a shuttle vector, which can propagate both in bacteria, such as, for example, E. coli (wherein the vector comprises an appropriate selectable marker and origin of replication) and be compatible for propagation in vertebrate cells, or integration in the genome of an organism of choice.

The nucleic acids may be introduced into tissues or host cells by any number of routes, including viral infection, microinjection, or fusion of vesicles. Jet injection may also be used for intra-muscular administration, as described by Furth et al. (1992), Anal Biochem 205:365-368. The nucleic acids may be coated onto gold microparticles, and delivered intradermally by a particle bombardment device, or “gene gun” as described in the literature (see, for example, Tang et al. (1992), Nature 356:152-154), where gold microprojectiles are coated with the DNA, then bombarded into skin cells. Expression vectors may be used to introduce the nucleic acids into a cell.

In one embodiment, the vectors of this invention may be fed directly to, injected into, the host organism containing the target gene. The vectors of this invention may be directly introduced into the cell (i.e., intracellularly); or introduced extracellularly into a cavity, interstitial space, into the circulation of an organism, introduced orally, etc. Methods for oral introduction include direct mixing of the vector with food of the organism. Physical methods of introducing the vectors include injection directly into the cell or extracellular injection into the organism of a solution comprising the vector. The vectors may be introduced in an amount, which allows delivery of at least one copy per cell. Higher doses (e.g., at least 5, 10, 100, 500 or 1000 copies per cell) of the vectors may yield more effective inhibition; lower doses may also be useful for specific applications.

In other embodiments, a hydrodynamic administration protocol is employed, and may be as described in Chang et al., J. Virol. (2001) 75:3469-3473; Liu et al., Gene Ther. (1999) 6:1258-1266; Wolff et al., Science (1990) 247: 1465-1468; Zhang et al., Hum. Gene Ther. (1999) 10:1735-1737: and Zhang et al., Gene Ther. (1999) 7:1344-1349, each of which represents an embodiment of this invention.

In other embodiments, delivery protocols of interest may include, but are not limited to: those described in U.S. Pat. No. 5,985,847, or 5,922,687, WO/11092;. Acsadi et al., New Biol. (1991) 3:71-81; Hickman et al., Hum. Gen. Ther. (1994) 5:1477-1483; or Wolff et al., Science (1990) 247: 1465-1468, and others, as will be appreciated by one skilled in the art.

The methods of this invention comprise the step of contacting a target cell with a vector of this invention. In one embodiment, the terms “contacting”, “contact” or “contacted” indicate, direct or, in another embodiment, indirect exposure of the cell to a vector, compound or composition comprising the vectors of this invention. It is envisaged that, in another embodiment, indirect supply to the cell may be via provision in a culture medium that surrounds the cell, or via parenteral administration in a body of a subject, whereby the vector ultimately contacts a cell via peripheral circulation (for further detail see, for example, Methods in Enzymology Vol. 1-317, Rubin and Dennis, eds, (1955-2003) and Current Protocols in Molecular Biology, Ausubel, et al, eds (1998), Molecular Cloning: A Laboratory Manual, Sambrook and Russell, eds., (2001), or other standard laboratory manuals). It is to be understood that any direct means or indirect means of intracellular access of a vector, or composition comprising the same of this invention represents an embodiment thereof.

In one embodiment, the target cell is contacted with a vector/composition comprising the same, of this invention, in vivo, in vitro or ex-vivo. In one embodiment, cells may be procured from a subject, contacted with a vector of this invention, and reintroduced into the subject In one embodiment, the cell is a stem or progenitor cell, and reintroduction into the subject may be followed, in another embodiment, by stimulation of differentiation of the contacted cell, in vivo.

In another embodiment, Cre recombinase is expressed at specific times during development.

In another embodiment, this invention provides for the generation of a non-human animal with reduced expression of a coding sequence, wherein the reduced expression is produced according to the methods, and/or utilizing the vectors of this invention.

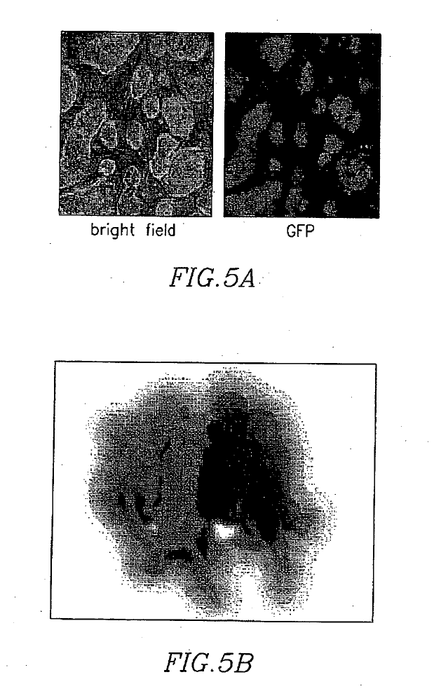

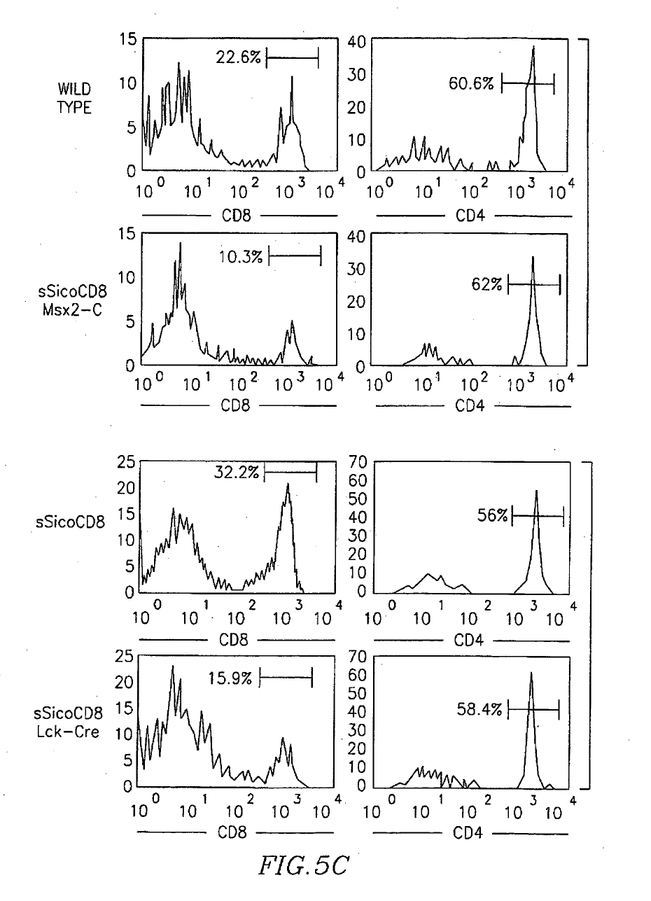

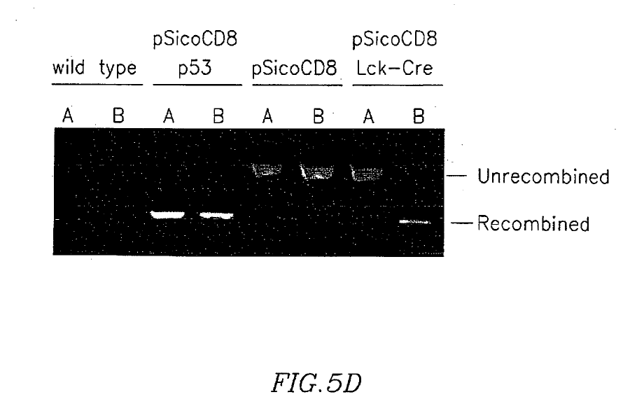

As exemplified further hereinunder, vectors comprising the mutant lox site containing a functional TATA box in its spacer sequence, provided Cre-regulated transcription and efficient processing of a normal-length shRNA, in vivo (FIG. 5). The pSico vector was demonstrated herein to achieve tissue-specific, conditional RNA interference in transgenic mice.

Transgenic mice, may, in one embodiment, be derived using the vectors/methods of this invention, according to Hogan, et al., “Manipulating the Mouse Embryo: A Laboratory Manual”, Cold Spring Harbor Laboratory (1988) which is incorporated herein by reference. Embryonic stem cells may, in another embodiment, be manipulated according to published procedures (Teratocarcinomas and embryonic stem cells: a practical approach, E. J. Robertson, ed., IRL Press, Washington, D.C., 1987; Zjilstra et al., Nature 342:435-438 (1989); and Schwartzberg et al., Science 246:799-803 (1989), each of which is incorporated herein by reference). Zygotes may be manipulated, in another embodiment, according to known procedures; for example see U.S. Pat. No. 4,873,191, Brinster et al., PNAS 86:7007 (1989); Susulic et al., J. Biol. Chem. 49:29483 (1995), and Cavard et al., Nucleic Acids Res. 16:2099 (1988), hereby incorporated by reference. Tetraploid blastocyst complementation may also be utilized to achieve non-human animals, which express the vectors of this invention, according to methods as exemplified herein, or, as are well known in the art.

In one embodiment, this invention provides a method of producing an animal genetically inactivated for a coding sequence, the method comprising contacting an embryonic stem cell with a vector of this invention which may be used for gene silencing, injecting the contacted embryonic stem cell to a blastocyst of an animal and obtaining an animal expressing the vector, whereby, following Cre-mediated recombination in the animal, the miRNA agent is expressed and reduces expression of the coding sequence, thereby being a method of producing an animal genetically inactivated for a coding sequence.

In another embodiment, this invention provides a method of producing an animal genetically inactivated for a coding sequence, the method comprising contacting a single cell embryo of an animal with a vector of this invention which may be used for gene silencing, and obtaining an animal expressing the vector, whereby, following Cre-mediated recombination in the animal, the miRNA agent is expressed and reduces expression of the coding sequence, thereby being a method of producing an animal genetically inactivated for a coding sequence.

In another embodiment, this invention provides a method for identifying a gene product involved in carcinogenesis, the method comprising obtaining the non-human animal of this invention, wherein conditionally reduced expression of a coding sequence has been achieved, wherein the coding sequence is for a gene product which is putatively involved in carcinogenesis, maintaining the animal under conditions stimulating, facilitating or promoting carcinogenesis, initiating or enabling Cre-mediated recombination in the animal and identifying the inhibition or suppression of carcinogenesis in the animal, wherein inhibition or suppression of carcinogenesis in the animal indicates said coding sequence is from a gene whose product is involved in carcinogenesis.

In another embodiment, the method of conditionally reducing expression of a coding sequence, as described and exemplified herein, may be therapeutic. In one embodiment, the term “therapeutic” refers to the fact that when in contact with a cell in a subject in need, provides a beneficial effect.

In one embodiment, the compositions/vectors and methods of conditionally reducing expression of a coding sequence of this invention prevent inappropriate expression of an encoded protein in a subject. Some examples include endogenous proteins which are mutated, and produces a non-functional protein, or an over-expressed protein, which in another embodiment, may be non-functional, or in another embodiment, pathogenic.

In one embodiment, the encoded protein may include cytokines, such as interferons or interleukins, or their receptors. According to this aspect of the invention, and in one embodiment, inappropriate expression patterns of cytokines may be altered to produce a beneficial effect, such as for example, a biasing of the immune response toward a Th1 type expression pattern, or a Th2 pattern in infection, or in autoimmune disease, wherein altered expression patterns may prove beneficial to the host. In these cases, and in one embodiment, conditionally reducing expression of the inappropriate or non-protective cytokine/receptor may be followed by delivery of an appropriate cytokine, or a vector/nucleic acid for expressing the same.

In another embodiment, the encoded protein may include an enzyme, such as one involved in glycogen storage or breakdown. In another embodiment, the encoded protein may include a transporter, such as an ion transporter, for example CFTR, or a glucose transporter, or other transporters whose inappropriate expression results in a variety of diseases. As described hereinabove, and in another embodiment, conditionally reducing expression of the encoded proteins, according to this aspect of the invention, may be followed by delivery of a wild-type protein, or a plasmid encoding same, or a mutated protein, which results in a therapeutic effect in the subject.

In another embodiment, the encoded protein may include a receptor, such as one involved in signal transduction within a cell. Some examples include as above, cytokine receptors, leptin receptors, transferring receptors, etc., or any receptor wherein altered expression results in inappropriate or inadequate signal transduction in a cell.

It is to be understood that any encoded protein, wherein conditionally reducing expression of the product is therapeutic to a subject is to be considered as part of this invention, and methods/vectors to provide wild-type or otherwise therapeutic versions of the encoded protein to the subject, following conditional reduction of expression of the mutated version, is to be considered as part of this invention, and embodiments thereof.

In another embodiment, the vectors/methods of this invention may be utilized to conditionally reduce expression of an oncogene, whose expression promotes cancer-related events In one embodiment, the conditionally reduced expression of oncogenes comprising ABLI, BCLI, BCL2, BCL6, CBFA2, CBL, CSFIR, ERBA, ERBB, EBRB2, ETSI, ETS1, ETV6, FOR, FOS, FYN, HCR, HRAS, JUN, KRAS, LCK, LYN, MDM2, MLL, MYB, MYC, MYCLI, MYCN, NRAS, PIM 1, PML, RET, SRC, TALI, TCL3, YES, or combinations thereof, may be effected via the vectors/compositions/methods of this invention. In another embodiment, vectors/methods of this invention may be utilized to conditionally reduce expression of a Prostate Tumor Inducing Gene, which may comprise in one embodiment, PTI-1, PTI-2, PTI-3 or combinations thereof.

In one embodiment, the vectors/methods of this invention may be utilized to conditionally reduce expression of genes whose products promote angiogenesis, such as, for example, and in one embodiment, VEGF, VEGF receptor, erythropoietin, or combinations thereof. In another embodiment, the coding sequence for which conditional reducing expression is desired may comprise a matrix metalloproteinase, wherein reduction of expression prevents, in one embodiment, metastasis of cancerous cells, or, in another embodiment, tissue necrosis in infectious or inflammatory diseases.

In another embodiment, the vectors/compositions/methods of this invention may be utilized to conditionally reduce expression of a mutated rhodopsin gene. Autosomal dominant retinitis pigmentosa (ADRP) is characterized by the substitution of histidine for proline at codon 23 (P23H) in their rhodopsin gene, resulting in photoreceptor cell death from the synthesis of the abnormal gene product. In one embodiment, P23H mutant mRNAs may be targeted for conditional reduction of expression.

In another embodiment, the vectors/compositions/methods of this invention may be utilized to reverse effects of high glucose on progression of diabetic retinopathy. High glucose environments can result in chronically increased nitric oxide (NO) activity, which leads to endothelial cell dysfunction and impaired blood retinal barrier integrity characteristic of diabetic retinopathy.

In one embodiment, NOS synthesis may be conditionally reduced, in a tissue specific manner, in another embodiment, via the use of miRNAs targeted against VEGF, iNOS, or eNOS using the vectors/compositions and methods, as described hereinabove. In another embodiment, glucose transporters may be similarly targeted for therapeutic purposes in diabetic retinopathy.

In another embodiment, the vectors/compositions and methods for reducing expression of a coding-sequence may be applied in a subject with a disease, where the disease may comprise, but is not limited to: muscular dystrophy, cancer, cardiovascular disease, hypertension, infection, renal disease, neurodegenerative disease, such as alzheimer's disease, parkinson's disease, huntington's chorea, Creuztfeld-Jacob disease, autoimniune disease, such as lupus, rheumatoid arthritis, endocarditis, Graves' disease or ALD, respiratory disease such as asthma or cystic fibrosis, bone disease, such as osteoporosis, joint disease, liver disease, disease of the skin, such as psoriasis or eczema, ophthalmic disease, otolaryngeal disease, other neurological disease such as Turret syndrome, schizophrenia, depression, autism, or stoke, or metabolic disease such as a glycogen storage disease or diabetes. It is to be understood that any disease whereby reduced expression of a particular protein, which can be accomplished via the use of the vectors or cells or compositions, or via the methods of this invention, is to be considered as part of this invention

In another embodiment, this invention provides a method of conditionally expressing a coding sequence in a target cell, the method comprising contacting the target cell with a vector comprising:
i. An RNA Polymerase III promoter downstream of a loxP site;
ii. An miRNA agent specific for said coding sequence, operatively-linked thereto; and
iii. A loxP site downstream of said miRNA agent;
   wherein the cell expresses the miRNA agent, thereby reducing expression of the coding sequence and whereby, following expression of the miRNA agent, cre-mediated recombination is enabled in the target cell, such that the miRNA agent is no longer expressed, thereby being a method of conditionally expressing a coding sequence in a target cell.

Specific, Cre-dependent re-expression of coding sequences was exemplified herein, such as, for example, re-expression of Npm, observed in pSicoR-Npm infected MEFs (FIGS. 4A, C).

In one embodiment, the Polymerase III promoter is a U6 promoter, or any RNA Polymerase III promoter, as described hereinabove, or as is known in the art. In another embodiment, the loxP site is a canonical loxP site, as described, and exemplified herein. In another embodiment, the miRNA agent is as described hereinabove, or as exemplified herein.

It is to be understood that any embodiment, or permutation thereof, described for a method/vector/composition hereinabove, in reference to conditionally reducing expression of a coding sequence, may be applied to that of the vectors/compositions or methods of conditionally expressing a coding sequence, and represent embodiments of this invention.

According to this aspect of the invention and in another embodiment, this invention provides a method of producing an animal genetically reactivated for a coding sequence, the method comprising contacting an embryonic stem cell with a vector for conditionally expressing a coding sequence, injecting the embryonic stem cell to a blastocyst of the animal, and obtaining an animal expressing the vector, whereby, following Cre-mediated recombination in the animal, the miRNA agent is no longer expressed and the coding sequence is expressed, thereby being a method of producing an animal genetically reactivated for a coding sequence.

In another embodiment, this invention provides a method of producing an animal genetically reactivated for a coding sequence, the method comprising contacting a single cell embryo of the animal a vector for conditionally expressing a coding sequence, and obtaining an animal expressing the vector, whereby, following Cre-mediated recombination in the animal, the miRNA agent is no longer expressed and the coding sequence is expressed, thereby being a method of producing an animal genetically reactivated for a coding sequence.

In one embodiment, conditional expression of the coding sequence is accomplished at a specific developmental stage. Such expression may be accomplished, in one embodiment, via delivery of a cre recombinase to a desired cell at a specific developmental stage, or in another embodiment, the cre recombinase is present in the cell, under the control of an inducible promoter, and cre expression is induced at a specific developmental stage. In another embodiment, conditional expression of the coding sequence is accomplished in specific tissues or cells, via similar methodology, or in another embodiment, via targeted delivery of a cre recombinase to a particular cell, such as, for example via delivery in a pseudotyped viral vector, which specifically infects a desired cell type.

In another embodiment, the coding sequence for which conditional expression is desired may comprise insulins, amylases, proteases, lipases, kinases, phosphatases, glycosyl transferases, trypsinogen, chymotrypsinogen, carboxypeptidases, hormones, ribonucleases, deoxyribonucleases, triacylglycerol lipase, phospholipase A2, elastases, amylases, blood clotting factors, UDP glucuronyl transferases, omithine transcarbamoylases, cytochrome p450 enzymes, adenosine deaminases, serum thymic factors, thymic humoral factors, thymopoietins, growth hormones, somatomedins, costimulatory factors, antibodies, colony stimulating factors, erythropoietin, epidermal growth factors, hepatic erythropoietic factors (hepatopoietin), liver-cell growth factors, interleukins, interferons, negative growth factors, fibroblast growth factors, transforming growth factors of the a family, transforming growth factors of the β family, gastrins, secretins, cholecystokinins, somatostatins, serotonins, substance P and transcription factors and enzymes (e.g., ACC synthases and oxidases, ACP desaturases and hydroxylases, ADP-glucose pyrophorylases, ATPases, alcohol dehydrogenases, amyloglucosidases, catalases, cellulases, chalcone synthases, chitinases, cyclooxygenases, decarboxylases, dextrinases, DNA and RNA polymerases, galactosidases, glucanases, glucose oxidases, granule-bound starch synthases, GTPases, helicases, hemicellulases, integrases, inulinases, invertases, isomerases, kinases, lactases, Upases, lipoxygenases, lyso/ymes, nopaline synthases, octopine synthases, pectinesterases, peroxidases, phosphatases, phospholipases, phosphorylases, phytases, plant growth regulator synthases, polygalacturonases, proteinases and peptidases, pullanases, recombinases, reverse transcriptases, RUBISCOs, topoisomerases, and xylanases); chemokines (e.g. CXCR4, CCR5), the RNA component of telomerase, vascular endothelial growth factor (VEGF), VEGF receptor, tumor necrosis factors nuclear factor kappa B, transcription factors, cell adhesion molecules, Insulin-like growth factor, transforming growth factor beta family members, cell surface receptors, RNA binding proteins (e.g. small nucleolar RNAs, RNA transport factors), translation factors, telomerase reverse transcriptase), or combinations thereof.

In another embodiment, the coding sequence for which conditional expression is desired may comprise a tumor suppressor gene, such as, for example, APC, BRCA 1, BRCA2, MADH4, MCC, NF 1, NF2, RB 1, TP53, WTI, or combinations thereof Conditional expression of these genes, in turn, may in one embodiment, suppress, or in another embodiment, diminish severity, or in another embodiment, prevent metastasis of a cancer.

In another embodiment, the coding sequence for which conditional expression is desired may comprise an immunomodulating protein, such as, for example, cytokines, chemokines, complement components, immune system accessory and adhesion molecules or their receptors, such as, for example, GM-CSF, IL-2, IL-12, OX40, OX40L (gp34), lymphotactin, CD40, and CD40L, interleukins 1 to 15, interferons alpha, beta or gamma, tumour necrosis factor, granulocyte-macrophage colony stimulating factor (GM-CSF), macrophage colony stimulating factor (M-CSF), granulocyte colony stimulating factor (G-CSF), chemokines such as neutrophil activating protein (NAP), macrophage chemoattractant and activating factor (MCAF), RANTES, macrophage inflammatory peptides MIP-1a and MIP-1b, complement components and their receptors, or an accessory molecule such as B7.1, B7.2, TRAP, ICAM-1, 2 or 3, cytokine receptors, OX40, OX40-ligand (gp34), or combinations thereof.

In another embodiment, the coding sequence for which conditional expression is desired may comprise a protein, which suppresses angiogenesis. Such a scenario is desirable in a number of disease states, including cancer, hemangiomas, glaucoma, and other diseases, as will be well known to one skilled in the art. In one embodiment, suppression of angiogenesis is accomplished via conditionally expressing an endostatin.

In another embodiment, the methods/vectors/compositions of this invention do not exhibit the limitation of causing constitutive gene silencing or gene expression, in all tissues According to this aspect of the invention, the methods of this allow for regulated expression of miRNA and thereby regulated expression of a desired coding sequence.

In another embodiment, this invention provides a method of identifying a tumor suppressor gene, the method comprising obtaining a non-human animal of this invention, which conditionally expresses a coding sequence, wherein the coding sequence is for a putative tumor suppressor, maintaining the animal under conditions promoting carcinogenesis, initiating or enabling cre-mediated recombination in the animal following carcinogenesis, and identifying inhibition or suppression of carcinogenesis in the animal, wherein inhibition or suppression of carcinogenesis in the animal indicates the coding sequence is from a tumor suppressor gene.

In another embodiment, and as exemplified herein with Dnmt1, the vectors of this invention may be used to determine the functional consequences of gene reactivation, and, in another embodiment, may facilitate “rescue” experiments in vivo. In one embodiment, the vectors of this invention used in vivo provide a means for mimicking the action of small molecule drugs designed to activate the proteins or pathways controlled by human disease genes. For example, and in one embodiment, conditional expression of tumor suppressor genes, according to the methods of this invention provide a means of identifying useful small molecules/drug targets, which impact cancer. In another embodiment, conditional expression of specific transporters may be mimicked for the design of similar small molecules, etc. as a means of identifying promising novel targets for drug development.

Because preparation of conditional RNAi constructs requires merely cloning of short synthetic DNA sequences, in one embodiment of this invention, a large number of conditional knock-down strains can be generated in parallel. In one embodiment, this approach is utilized for large-scale projects aimed at the characterization of genetic pathways or at the validation of candidate target genes identified through gene-profiling screenings. For example, and in another embodiment, gene expression profiling using mouse cancer models, which typically yields numerous genes that distinguish tumor from normal tissue, if assessed using conventional or conditional knock-out strategies, then only a small fraction of these genes are evaluated for functional relevance to tumorigenesis, while the methods of the present invention allow for conditional systems such as pSico to greatly reduce the time, cost and effort required to perform experiments of this magnitude.

In another embodiment, this invention provides for kits for conditional reduction of expression, or conditional expression of a coding sequence, comprising one or more containers filled with one or more of the ingredients of the aforementioned vectors, or compositions of the invention.

The vectors of the invention may be employed, in another embodiment, in combination with a non-sterile or sterile carrier or carriers for administration to cells, tissues or organisms, such as a pharmaceutical carrier suitable for administration to an individual. Such compositions comprise, for instance, a media additive or a therapeutically effective amount of a recombinant virus of the invention and a pharmaceutically acceptable carrier or excipient. Such carriers may include, but are not limited to, saline, buffered saline, dextrose, water, glycerol, and combinations thereof. The formulation should suit the mode of administration.

The vectors or compositions of the invention may be employed alone or in conjunction with other compounds, such as additional therapeutic compounds.

The pharmaceutical compositions may be administered in any effective, convenient manner including, for instance, administration by intravascular (i.v.), intramuscular (i.m.), intranasal (i.n.), subcutaneous (s.c.), oral, rectal, intravaginal delivery, or by any means in which the recombinant virus/composition can be delivered to tissue (e.g., needle or catheter). Alternatively, topical administration may be desired for insertion into epithelial cells. Another method of administration is via aspiration or aerosol formulation.

For administration to mammals, and particularly humans, it is expected that the physician will determine the actual dosage and duration of treatment, which will be most suitable for an individual and can vary with the age, weight and response of the particular individual.

The following examples are presented in order to more fully illustrate some embodiments of the invention. They should, in no way be construed, however, as limiting the scope of the invention.

### EXAMPLES

### Example 1

### Functional TATA-Lox-Modified U6 Promoter

### Materials and Methods

Generation of Plasmids

To generate pSico the lox-CMV-GFP-lox cassette was removed from lentilox 3.7 (pLL3.7) (Rubinson, D. A., et al. (2003) Nat Genet 33, 401-6) by digesting with BfuAI and PciI, followed by filling-in and religation. The first TATAlox followed by the terminator and by an EcoRI was inserted in the resulting plasmid by PCR-mediated mutagenesis using the following oligos:

(SEQ ID NO: 1)

pSico6Eco: GAATTCAACGCGCGGTGACCCTCGAGG

(SEQ ID NO: 2)

pSico6: ASAAAAAACCAAGGCTTATAACTTCGTATAATTTATACTATA
CGAAGTTATAATTACTTTACAGTTACCC

To insert the second TATAlox preceded by a NotI site the resulting plasmid was digested with EcoRI and XhoI and ligated to the following annealed oligos:

(SEQ ID NO: 3)

TATALOX F:
AATTCGAOAGGCGGCCGCATAACTTCGTATAGTATAAATTATACGAAGTT
ATAAGCCTTGTTAACGCGCGGTGACCC

(SEQ ID NO: 4)

TATALOX R:
TCGAGGGTCACCGCGCGTTAACAAGGCTTATAACTTCGTATAATTTATAC
TATACGAAGTTATGCGGCCGCCTCTCG

The resulting construct was finally digested with EcoRI and NotI and ligated to an EcoRI-CMV-GFP-NotI cassette amplified from pLL3.7.

The luciferase shRNA coding oligos were cloned by ligating to the HpaI/XhoI digested vector the following annealed oligos:

(SEQ ID NO: 5)

Luc sense:
TGAGCTGTTTCTGAGGAGCCTTCAAGAGAGGCTCCTCAGAAACAGCTCTT
TTTTC.

(SEQ ID NO: 6)

Luc reverse:
TCGAGAAAAAAGCTGGATAATGCCAGGCAGTCTCTTGAACTGCCTGGCAT
TATCCAGCA

Luciferase Activity

293T cells were co-transfected in 12 well plates using FUGENE 6 with the appropriate shRNA vectors together with pGL3control and pRLSV40. Total amount of transfected DNA was 500 ng/well. Firefly and Renilla luciferase activity were measured 36 hours after transfection using the dual reporter kit (Promega) according to the manufacturer's instruction. All experiments were performed in triplicate.

### Results

The U6 promoter has been widely used to drive the expression of shRNAs and a U6 based lentiviral vector for the generation transgenic mice has been recently described. To control its activity in a Cre-dependent manner, the U6 promoter was modified by inserting a Lox-STOP-Lox cassette in its sequence. Analogously to other RNA Polymerase III promoters, the U6 promoter is extremely compact, its functional elements consisting in a TATA box, a proximal sequence element (PSE) and a distal sequence element (DSE) (FIG. 1A). Mutagenesis experiments have demonstrated that while the DSE is largely dispensable for transcriptional activity, the PSE and the TATA box are absolutely required. In addition, the spacing between the PSE and the TATAbox (17 nucleotides) and between the TATA box and the transcription start site (22 nucleotides) is critical and even small changes have been shown to severely impair promoter activity. To be effective, the Lox-stop-Lox element must therefore be positioned either between the PSE and the TATA box or between the TATA box and the transcription start site. Furthermore, after Cre-mediated recombination, the normal spacing between these elements must be restored. These considerations clearly prevent the utilization of a classic lox-STOP-lox cassette since after Cre mediated excision the residual lox site (34 nucleotide) would necessarily increase the PSE-TATA or the TATA-start site spacing, thus resulting in a non-functional promoter (FIG. 1B).

To overcome this limitation a novel, bifunctional lox site (indicated from now on as “TATA-lox”) was generated that in addition to retaining the ability to undergo Cre-mediated recombination, contained a functional TATA box in its spacer region (FIG. 1C). TATA-lox had a nucleotide sequence corresponding to:

ATAACTTCGTATAGTATAAATTATACGAAGTTAT (SEQ ID NO: 7), and shares substantial identity with the canonical LoxP site, which has a nucleotide sequence corresponding to:

| | |
|---|---|
| ATAACTTCGTATAGCATACATTATACGAAGTTAT. | (SEQ ID NO: 8) |

The TATA-lox can replace the TATA box site in the U6 promoter without altering the spacing between PSE, TATA and transcriptional start site (FIG. 1C). Thus, the wild-type U6 promoter has a nucleotide sequence of 69 nucleotdes, with a sequence corresponding to:

(SEQ ID NO: 9)

CTCACCCTAACTGTAAAGTAATTGTGTGTTTTGAGACTATAAATATCCCT
TGGAGAAAAGCCTTGTTTG.

The U6 promoter, modified to incorporate TATA-lox, designated as cU6, has a nucleotide sequence corresponding to:
CTCACCCTAACTGTAAAGTAATTATAACTTCGTATAGTATAAATTATA CGAAGTTATAAGCCTTGTTTG (SEQ ID NO: 10), and being 69 nucleotides long.

To verify that this resulted in a functional promoter, the ability of the cU6 promoter to drive the expression of an shRNA directed against the firefly luciferase mRNA was determined, in comparison to wild-type U6. Both promoters caused similar reduction of luciferase activity (FIG. 1D) indicating that the TATA-lox can substitute the TATA box without significantly impairing its transcriptional activity.

### Example 2

### Conditional shRNA Expression with TATA-Lox U6 Promoters

### Materials and Methods

Plasmids:

The CMV-GFP cassette was amplified from pLL3.7 (Rubinson, D. A., et al, (2003) Nat Genet 33, 401-6.)

The complete sequence of the cU6-CMV-EGFP-TATAlox-shRNA:

(SEQ ID NO: 11)

GATCCGACGCCGCCATCTCTAGGCCCGCGCCGGCCCCCTCGCACAGACTT
GTGGGAGAAGCTCGGCTACTCCCCTGCCCCGGTTAATTTGCATATAATAT
TTCCTAGTAACTATAGAGGCTTAATGTGCGATAAAAGACAGATAATCTGT
TCTTTTTAAIACTAGCTACATTTTACATGATAGGCTTGGATTTCTATAAG
AGATACAAATACTAAATTATTATTTTAAAAAACAGCACAAAAGGAAACTC
ACCCTAACTGTAAAGTAATTATAACTTCGTATAGTATAAATTATACGAAG
TTATAAGCCTTGGTTTTTTGAATTCCGTATTACCGCCATGCATTAGTTAT
TAATAGTAATCAATTACGGGGTCATTAGTTCATAGCCCATATATGGAGTT
CCGCGTTACATAACTTACGGTAAATGGCCCGCCTGGCTGACCGCCCAACG
ACCCCCGCCCATTGACGTCAATAATGACGTATGTTCCCATAGTAACGCCA
ATAGGGACTTTCCATTGACGTCAATGGGTGGAGTATTTACGGTAAACTGC
CCACTTGGCAGTACATCAAGTTGTATCATATGCCAAGTACGCCCCCTATT
GACGTCAATGACGGTAAATGGCCCGCCTGGCATTATGCCCAGTACATGAC
CTTATGGGACTTTCCTACTTGGCAGTACATCTACGTATTAGTCATCGCTA
TTACCATGGTGATGCGGTTTTGGCAGTACATCAATGGGCGTGGATAGCGG
TTTGACTCACGGGGATTTCCAAQTCTCCACCCCATTGACGTCAATGGGAG
TTTGTTTTGGCACCAAAATCAACGGGACTTTCCAAAATGTCGTAACAACT
CCGCCCCATTGACGCAAATGGGCGGTAGGCGTGTACGGTGGGAGGTCTAT
ATAAGCAGAGCTGGTTTAGTGAACCGTCAGATCCGCTAGCGCTACCGGTC
GCCACCATGGTGAGCAAGGGCGAGGAGCTGTTCACCGGGGTGGTGCCCAT
CCTGGTCGAGCTGGACGGCGACGTAAACGGCCACAAGTTCAGCGTGTCCG
GCGAGGGCGAGGGCGATGCCACCTACGGCAAGCTGACCCTGAAGTTCATC
TGCACCACCGGCAAGCTGCCCGTGCCCTGGCCCACCCTCGTGACCACCCT
GACCTACGGCGTGCAGTGCTTCAGCCGCTACCCCGACCACATGAAGCAGC
ACGACTTCTTCAAGTCCGCCATGCCCGAAGGCTACGTCCAGGAGCGCACC
ATCTTCTTCAAGGACGACGGGAACTACAAGACCCGCGCCGAQGTGAAGTT
CGAGGGCGACACCCTGGTGAACCGCATCGAGCTGAAGGGCATCGACTTCA
AGGAGGACGGCAACATCCTGGGGCACAAGCTGGAGTACAACTACAACAGC
CACAACGTCTATATCATGGCCGACAAGCAGAGAACGGCATCAAGGTGAAC
TTCAAGATCCGCCACAACATCGAGGACGGCAGCGTGCAGCTCGCCGACCA
CTACCAGCAGAACACCCCCATCGGCGACGGCCCCGTGCTGCTGCCCGACA
ACCACTACCTGAGCACCCAGTCCGCCCTGAGCAAAGACCCCAACGAGAAG
CGCGATCACATGGTCCTGCTGGAGTTCGTGACCGCCGCCGGGATCACTCT
CGGCATGGACGAGCTGTACAAGTAGCGGCCGCATAACTTCGTATAGTATA
AATTATACGAAGTTATAAGCCTTGTTTGAGCTGTTTCTGAGGAGCCTTCA
AGAGAGGCTCCTCAGAAACAGCTCTTTTTTC.

The shRNA encoding sequence was:

(SEQ ID NO: 12)

TGAGCTGTTTCTGAGGAGCCTTCAAGAGAGGCTCCTCAGAAACAGCTCTT
TTTTC.

Cells and Infection:

293T cells were transfected with reporter plasmids expressing firefly luciferase and renilla luciferase, pGL3 promoter and pRLSV40 (Promega), concurrently with equal amounts of wildtype U6 promoter constructs and the TATA-lox U6 promoter construct, with/out driving the expression of an shRNA directed against the luciferase gene for 36 hours. Cells were lysed, and the ratio between firefly and renilla luciferase activity was measured.

### Results

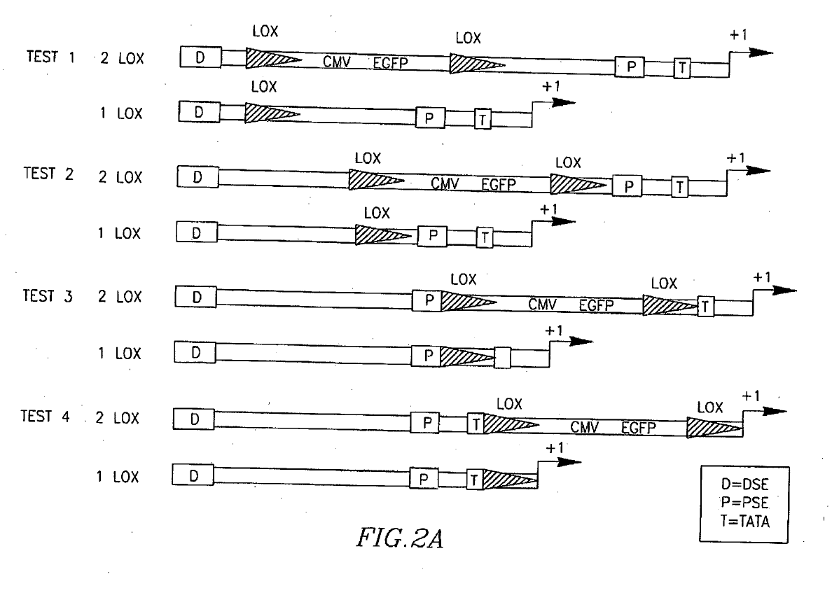

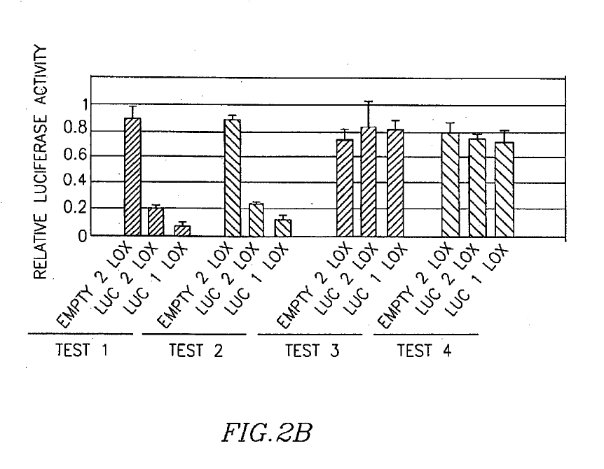

A CMV-EGFP stop/reporter cassette was placed between two TATA-lox sites, such that Cre-mediated recombination resulted in excision of the cassette, with reconstitution of a functional U6 promoter containing a TATA-lox in place of the TATA box (FIG. 2A). To prevent unwanted transcription downstream of the first TATA-lox, a run of six “T” was positioned immediately upstream the CMV promoter to serve as a “STOP” signal.

To assess whether the presence of the lox-STOP/reporter-lox cassette is sufficient to prevent transcription of a downstream shRNA, the ability of the conditional U6 promoter to drive the expression of a luciferase shRNA before and after Cre-mediated recombination was compared. As shown in FIG. 2A, while the 2 lox construct (prior to Cre-mediated recombination) did not induce any significant decrease in luciferase gene expression, the 1 lox construct (following Cre-mediated recombination) caused a dramatic reduction of the firefly/renilla luciferase ratio, indicating that the lox-CMV-GFP-lox cassette efficiently inactivated the U6 promoter.

### Example 3

### Conditional Endogenous Gene Silencing with Lentiviral Vectors Containing TATA-Lox U6 Promoters Driving shRNA Expression

### Materials and Methods

Generation of a Self-Inactivating Lentiviral Vector Containing a Conditional TATA-lox U6:

To generate pSico the lox-CMV-GFP-lox cassette was removed from lentilox 3.7 (pLL3.7) (Rubinson, supra) by digesting with BfuAI and PciI followed by filling-in and religation. The first TATAlox followed by the terminator and by an EcoRI was inserted in the resulting plasmid by PCR-mediated mutagenesis using the following oligos:

(SEQ ID NO: 1)

pSico6Eco GAATTCAACGCGCGGTGAGCCTCGAGG

(SEQ ID NO: 13)

pSico6 AS AAAAAACCAAGGCTTATAACTTCGTATAATTTATACTATA
CGAAGTTATAATTACTTTACAGTTACCC.

To insert the second TATAlox preceded by a NotI site the resulting plasmid was digested with EcoRI and XhoI and ligated to the annealed oligos, TATALOX F (SEQ ID NO: 3) (Example 1), and TATALOXR (SEQ ID NO: 4) (Example 1).

The resulting construct was finally digested with EcoRI and NotI and ligated to an EcoRI-CMV-GFP-NotI cassette amplified from pLL3.7.

Adenoviral Vectors:

Recombinant Adenoviral stocks were purchased from the Gene Transfer Vector Core facility of University of Iowa College of Medicine. Infections were performed using 100 plaque-forming units of virus per cell.

Subcloning of p53 siRNA Into pSico and pSico R:

Oligos designed to knockdown the mouse p53 gene (sense: TGTACTCTCCTCCCCTCAATTTCAAGAGAATTGAGGGGAGGAGAGTA CTTTTTTC (SEQ ID NO: 14, and antisense: TCGAGAAAAAAGTACTCTCCTCCCCTCAATTCTCTTGAAATTGAGGGG AGGAGAGTACA (SEQ ID NO: 15), designated p53 siRNA, were annealed and cloned in HpaI/XhoI digested psico, pSicoR and lentilox 3.7.

MEF Infection with Lentiviral Vectors:

p53 R270H/− mouse embryo fibroblasts (MEF) were infected with the indicated lentiviruses and were sorted by FACS for GFP positivity. GFP positive, MEF cells were then infected with Adeno empty or Adeno Cre. Four days post infection, genomic DNA was extracted and a PCR reaction was performed to amplify the recombined and unrecombined viral DNA, with primers used for loopout, forward: CCCGGTTAATTTGCATATAATATTTC (SEQ ID NO: 16), and reverse:

CATGATACAAAGGCATTAAAGCAG (SEQ ID NO: 17), at PCR conditions of 32 cycles at 94° C., 30 seconds, 56° C. 1 minute, and 72° C. 2 minutes. GFP positive, MEF cells were also visualized by epifluorescence microscopy.

P53 Gene Silencing:

MEF's expressing high basal levels of a p53 point mutant (R270H), which is a transcriptionally inactive, p53 allele (K. Olive and T. Jacks, submitted for publication) were infected with the vectors. One week later, the sorted, GFP-positive, MEF's were super-infected with empty adenovirus (Ad) or with Ad-Cre and the expression of the shRNA against p53 was examined, 4 days later, by Northern blots probing for the presence of the siRNA, using the mouse p53 coding sequence. 15 μg of total RNA extracted from the MEFs was separated on a 15% denaturing polyacrilamide gel, transferred to a nitrocellulose filter and hybridized to a radio-labeled 19-mer corresponding to the sense strand of the p53 shRNA (GTACTCTCCTCCCCTCAAT). Equal RNA loading was assessed by ethidium bromide staining of the upper part of the gel.

Antibodies, Chemicals and Western Blotting

Anti alpha-tubulin antibody was from Sigma, the p53 antibody was provided by Kristian Helin All antibodies used were mouse monoclonal. Doxorubicin and doxycycline were obtained from Sigma.

For western blotting cells were lysed in a buffer containing 1% TritonX-100, 10 mM TrisCi and 140 mM NaCl and a protease inhibitor cocktail (SIGMA). Proteins were resolved by SDS-PAGE, transferred to a filter, blocked overnight in 5% fat-free milk in TBS 0.1% Tween (TBS-T). After 1 h incubation with the primary antibody filters were washed in TBS-T, incubated 30 minutes with the appropriate HRP-conjugated secondary antibody, washed 3 times in TBS-T and processed using the ECL plus kit and exposed to film. Western blot analysis, probing for p53 protein expression was evaluated, as well, in these cells.

Immunocytochemistry

Cells plated on glass coverslips that had been pre-incubated with 0.1% gelatin in PBS at 37° C. for 30 minutes were fixed in 4% parafornaldehyde (in PBS) for 10 minutes, washed with PBS and permeabilized by incubating in PBS 0.1% Triton X-100 for 10 minutes at room temperature. To prevent non-specific binding of the antibodies, cells were then incubated with PBS in the presence of 5% Bovine Serum Albumin (BSA) for 30 minutes. The coverslips were then gently deposited, face down, on 100 μl of primary antibody diluted in PBS 5%BSA. After one-hour, coverslips were washed three times with PBS (5 minutes per wash). Cells were then incubated 30 minutes at RT with the appropriate secondary antibody Cy3 (Amersham), Alexa 488- or Alexa 350-conjugated (Molecular Probes). Coverslips were mounted in a 90% glycerol solution containing diazabicyclo-(2.2.2)octane antifade (Sigma) and examined by fluorescence microscopy. Images were further processed with the Adobe Photoshop software (Adobe).

p53 Functional Analysis:

Wild type MEFs were infected with pSico p53, pSicoR p53 or with pSico Luc and super-infected with Ad or Ad-Cre. Infected MEF's were either mock treated or treated with 1 μg/ml doxorubicin. pSico Luc and pSico p53 were administered doxorubicin, 4 days post Adeno infection, while treatment of pSicoR p53 and control cells was performed 10 days post Adeno infection Twelve hours post doxorubicin treatment, cell cycle profile and p53 protein levels were assayed by cytofluorimetry and Western blotting, respectively. For Western blots, whole cell lysates were prepared from the cells, which were separated by PAGE and immunoblotted against p53 and tubulin

Flow Cytometry

10⁶ cells were fixed in 70% ethanol, washed in PBS and resuspended in 20 μg/ml Propidium Iodide (SIGMA), 200 μg/ml RNAseA in PBS. Acquisition of samples was performed on a FACScan flow cytometer, and the data were analyzed with CELLQuest software (BD Immunocytometry Systems, San Jose, Calif.).

### Results

The conditional U6 cassette was inserted into a self-inactivating lentiviral vector backbone derived from lentilox 3.7 {Rubinson, 2003}, in order to allow for the efficient generation of conditional knock-down mice and cell lines. The resulting plasmid was named “pSico” (plasmid for stable RNA interference, conditional) (FIG. 2B) A vector, pSicoR (pSico Reverse) (FIG. 2C) that allows Cre-mediated inactivation of the U6 promoter was also generated, to extend the potential applications of lentivirus-mediated RNAi. The CMV GFP cassette is placed downstream of the shRNA in pSicoR, with one lox site placed between the DSE and the PSE in the U6 promoter, and the second lox site positioned immediately downstream of the GFP coding sequence. Cells infected with pSicoR produce the shRNA until a Cre-mediated recombination removes the whole U6-shRNA-CMV-GFP unit. The CMV-GFP cassette in both pSico and pSicoR allowed the ready identification of infected cells and was lost upon Cre mediated-recombination.

pSico, which contains two TATA-lox sites, as well as pSicoR, and lentilox 3.7 (Rubinson et al. 2003), which contains a CMV-GFP cassette flanked by lox sites (positive control), were tested for their abitlity to undergo efficient Cre-mediated recombination. Mouse embryo fibroblasts (MEF) were infected with lentilox 3.7, pSico or pSicoR and one week later GFP positive cells were super-infected with an empty Adenovirus or with an Adenovirus expressing the Cre recombinase (AdenoCre). pSico recombined with an efficiency similar to that of pSicoR and lentilox 3.7, indicating that the TATA-lox is a good substrate for the Cre enzyme (FIGS. 3A and 3B).

In order to determine whether pSico and pSicoR conditionally silenced endogenous genes, MEF's expressing high basal levels of a p53 point mutant were infected with the vectors, which contained shRNA directed against the p53 gene. Cre expression in the MEF's resulted in expression of the p53 shRNA, in pSico, or its absence in pSicoR, as demonstrated by Northern blot analysis (FIG. 3C). Cre expression induced p53 shRNA was observed in cells infected with pSico p53, while no detectable shRNA was observed in the same cells in the absence of previous Cre expression. The size of the processed RNA (21-24 nucleotides) was identical in cells infected with 113.7 p53, pSicop53 or pSicoR p53, indicating that the presence of the TATAlox in pSico does not qualitatively affect shRNA production. In addition, the amount of p53 siRNA in cells infected with pSicop53 and Cre was even higher than in the 113.7 and pSicop53R, (FIG. 3C, compare lanes 5 with lanes 3 and 6), a finding that suggests that the TATA-lox carrying U6 promoter might be more transcriptionally active than the wild type counterpart. Finally, as expected, Cre lead to almost complete disappearance of the p53siRNA in pSicoR p53 infected cells.

Cre expression lead to drastic reduction of p53 protein levels in pSicop53 infected cells, while it restored p53 levels in cells infected with pSicoR p53 (FIG. 3D, Western results). A small but significant increase in p53 siRNA and p53 knockdown was demonstrated, following Cre expression in cells infected with 113.7 p53 (FIG. 3C and 3D, lanes 2 and 3). This could reflect interference between the CMV and the U6 promoter since in 113.7 the foxed CMV-GFP cassette is immediately downstream the U6 promoter.

Wild type MEFs infected with pSico p53, pSicoR p53 or with pSico Luc, treated with 1μg/ml doxorubicin (a DNA damaging compound that induces G1 and S phase arrest in a p53-dependent manner), in cells expressing Cre, demonstrated that Cre expression in cells infected with pSico p53 was sufficient to lead to loss of p53 function (FIG. 3E, F). Cells infected with pSico Luc showed significant p53 induction and G1-S arrest in response to doxorubicin, regardless of whether they had previously been infected with Ad or Ad-Cre. In contrast, Ad-Cre infection of cells previously infected with pSicopS3 led to near complete inhibition doxorubicin-induced p53 induction and G1-S arrest. pSicoR p53 infected cells showed only modest rescue of doxorubicin-induced p53 activation and G1 arrest 4 days after Cre infection (data not shown), while the rescue was practically complete ten days post-Cre (FIG. 3 E, F), where the delayed kinetic may be in response to the need for the p53 shRNA/siRNA already present at the time of Cre expression to be diluted out and degraded.

### Example 4

### Muliple Genes Conditionally Silenced with Lentiviral Vectors Containing TATA-Lox U6 Promoters Driving shRNA Expression

### Materials and Methods

Subcloning of NPM siRNA and DNMT-1 siRNA Into pSico and pSico R:

Oligos designed to knockdown the nucleolar protein nucleophosmin (NPM) were as follows: (NPM sense:

(SEQ ID NO: 18)

TGGCTGACAAAGACTATCACTTCAAGAGAGTGATAGTCTTTGTCAGCCTT
TTTTC
and

(SEQ ID NO: 19)

NPM antisense: TCGAGAAAAAAGGCTGACAAAGACTATCACTCTCT
TGAAGTGATAGTCTTTGTCAGCCA.

Oligos designed to knockdown the DNA methyltransferase DNMT-1 gene were as follows: (Dnmt1 sense: TGAGTGTGTGAGGGAGAAATTCAAGAGATTTCTCCCTCACACACTCTT TTTTC (SEQ ID NO: 20) and Dnmt1 Antisense:

TCGAGAAAAAAGAGTGTGTGAGGGAGAAATCTCTTGAATTTCTCCCT CACACACTCA (SEQ ID NO: 21). The oligos were designated NPM and DNMT-1 siRNA, respectively, and were cloned in pSico, pSicoR and in lentilox 3.7.

NPM Gene Silencing:

MEFs were infected with the indicated lentiviruses, GFP positive cells were sorted and superinfected with empty Adenovirus or AdenoCre. 1 week later, whole cell lysates were separated by PAGE subjected to western blotting against NPM and tubulin. B. Embrionic stem cells carrying a Tetracycline inducible Cre [C. Beard and R. Jaenisch, unpublished] were infected with the indicated lentiviruses. GFP positive single clones were isolated, expanded, and split in two 35 mm wells and either left untreated or incubated with 10 μg/ml doxicycline for 1 week. Immunoblot analysis was performed as described above. Immunofluorescence microscopy analysis of MEFs infected with pSico NPM or pSicoR NPM was conducted, one-week post infection with empty Adenovirus or AdenoCre. Cells were plated on glass coverslips, fixed and probed with anti NPM antibody, while nuclei were stained with DAPI.

and the anti-Npm was a gift from Pier Giuseppe Pelicci and Emanuela Colombo.

DNMT-1 Gene Silencing:

DNA was isolated from the indicated ES cell lines To assess the levels of DNA methylation, genomic DNA was digested with HpaII, and hybridized to pMR150 as a probe for the minor satellite repeats (Chapman, V., Forrester, L., Sanford, J., Hastie, N. & Rossant, J. (1984) Nature 307, 284-6). For the methylation status of imprinted loci, a bisulfite conversion assay was performed using the CpGenome DNA modification kit (Chemicon) using PCR primers and conditions described previously (Lucifero, D., Mertineit, C., Clarke, H. J., Bestor, T. H. & Trasler, J. M. (2002) Genomics 79, 530-8.). PCR products were gel purified, digested with BstUI and resolved on a 2% agarose gel.

### Results

Gene silencing of two additional endogenous genes, namely the abundant and ubiquitously expressed nucleolar protein nucleophosmin (NPM) and the DNA methyltransferase DNMT-1 genes were tested as well. siRNA directed agains NPM and DNMT-1 were subcloned into pSico and pSicoR, and conditionally silenced gene expression, in a Cre-dependent fashion was similarly demonstrated in mammalian cells (FIG. 4).

Npm is a putative tumor suppressor gene involved in a number of chromosomal translocations associated with human leukemias. It has been shown to physically and functionally interact with the tumor suppressors p19ARF and p53. Specific, Cre-dependent knock-down of Npm was observed in both MEFs and embryonic stem (ES) cell clones infected with pSico-Npm (FIGS. 4). The opposite effect, Cre-dependent re-expression of Npm, was observed in pSicoR-Npm infected MEFs (FIGS. 4A, C).

The characterization of ES cells mutant for Dnmt1 has been previously reported, and demonstrated that Dnmt1 is required for genome-wide maintenance of cytosine methylation (Li, E., Bestor, T. H. & Jaenisch, R. (1992) Cell 69, 915-26). Dnmt1-deficient ES cells are viable and proliferate normally despite substantial loss of methylation; however, they die upon differentiation While re-expression of the Dnmt1 cDNA in these cells leads to re-methylation of bulk genomic DNA and non-imprinted genes, the methylation pattern of imprinted loci cannot be restored without germ-line passage pSico-Dnmt1 and pSicoR-Dnmt1 recapitulated this phenomenon. pSico-Dnmt1 infected ES cells underwent significant loss of CpG methylation of minor satellites (FIG. 4D) and of two imprinted genes tested (FIG. 4E) upon Cre induction. Importantly, the reacquisition of DNA methylation at minor satellites sequences, but not at imprinted loci in pSicoR-Dnmt1 after Cre-mediated recombination confirms previous results obtained with re-expression of Dnmt1. This further illustrates the potential for application of the pSicoR vector in vitro and in vivo to perform “rescue” experiments.

### Example 5

### In Vivo Conditional Gene Silencing

### Materials and Methods

Generation of the pSicoON CD8 and pSicoOFF CD8 Constructs

CD8 siRNA was as published (Rubinson, supra). To generate pSicoR CD8, the 5′ loxP site present in pLL3.7 was removed by digesting with XhoI and NotI and replaced with a diagnostic BamHI site using the following annealed oligos: Lox replace for TCGAGTACTAGGATCCATTAGGC (SEQ ID NO: 22) and Lox replace rev GGCCGCCTAATGGATCCTAGTAC (SEQ ID NO: 23).

A new lox site was inserted 18 nucleotides upstream of the proximal sequence element (PSE) in the U6 promotor by PCR-mediated mutagenesis.

ES Cells Manipulation, Generation of Chimeras and Tetraploid Complementation

V6.5 ES cells were cultivated on irradiated MEFs in DME containing 15% fetal calf serum, Leukemia Inhibiting Factor (LIF), Penicillin/Streptomycin, L-Glutamine, and non-essential aminoacids. MEFs were cultivated in DME 10% Fetal Calf Serum supplemented with L-Glutamine and Penicillin/Streptomycin. The derivative of V6.5 containing a doxycycline-inducible Cre transgene in the collagen locus was accomplished (C. Beard and R. Jaenisch, unpublished data).

B6D2F2 diploid blastocysts and B6D2F2 tetraploid blastocysts were generated and injected with ES cells as previously described (Eggan, K., et al., (2001) Proc Natl Acad Sci USA 98, 6209-14). Tetraploid blastocyst-derived animals were delivered by cesarean -section on day 19.5 post-coitum and fostered to lactating BALB/c mothers. Alternatively day 14.5 embryos were surgically removed to generate MEFs following standard procedure. Msx2-Cre mice (Sun, X., et al., (2000) Nat Genet 25, 83-6) were received from G. Martin and Lck-Cre mice (Hennet, T., et al., (1995) Proc Natl Acad Sci USA 92, 12070-4) were obtained from Jackson Laboratories.

Southern Blot and Methylation Analysis

DNA was isolated from the indicated ES cell lines. To assess the levels of DNA methylation, genomic DNA was digested with HpaII, and hybridized to pMR150 as a probe for the minor satellite repeats (Chapman, V., et al. (1984) Nature 307, 284-6). For the methylation status of imprinted loci, a bisulfite conversion assay was performed using the CpGenome DNA modification kit (Chemicon) using PCR primers and conditions described previously (Lucifero, D., et al. (2002) Genomics 79, 530-8). PCR products were gel purified, digested with BstUI and resolved on a 2% agarose gel.

Flow Cytometry

To assess expression of CD4 and CD8 in the chimeric mice, single cells suspensions of splenocytes were blocked with anti-CD16/CD32 for 10 min on ice. After blocking, the cells were incubated with phycoerthrin-conjugated anti-CD8, allophycocyanin conjugated anti-CD4, and PerCPCy5.5 conjugated anti-CD3 for 20 min at 4° C. (BD Pharmingen, San Diego, Calif.). Acquisition of samples was performed on a FACScan flow cytometer, and the data were analyzed with CELLQuest software (BD Immunocytometry Systems, San Jose, Calif.). Plots were gated on CD3+ cells

### Results

ES cells were infected with pSico-CD8 (FIG. 5A), which was designed to inhibit expression of the T-lymphocyte cell surface marker CD8. Three pSico-CD8 ES clones were used to generate chimeric mice and transmission of the pSico-CD8 transgene to the progeny was observed for two of them. All transgenic mice were easily identified by macroscopic GFP visualization (FIG. 5B), although some variability in the extent and distribution of GFP expression among littermates was observed. Importantly, all transgenic mice presented normal amounts of CD4+ and CD8+ lymphocytes and were apparently normal and fertile, indicating that the presence of the non-expressing pSico-CD8 transgene prior to Cre activation did not affect CD8 expression and was compatible with normal mouse development.

In order to achieve either global or tissue-specific activation of the CD8 shRNA, pSico-CD8 chimeras were crossed to Msx2-Cre or Lck-Cre transgenic mice that express Cre in the oocyte (Sun, X., et al. (2000) Nat Genet 25, 83-6; Gaudet, F., et al. (2004) Mol Cell Biol 24, 1640-8) or under the control of a T cell-specific promoter (Hennet, T., et al. (1995) Proc Natl Acad Sci USA 92, 25 120704), respectively. FACS analysis demonstrated that pSico-CD8;Lck-Cre and pSico-CD8;Msx2-Cre mice had a specific reduction in splenic CD8+, but not CD4+ T-lymphocytes as compared to controls (FIG. 5C). pSico-CD8; Msx2-Cre progeny showed complete recombination of the pSicoCD8 transgene and lacked detectable GFP expression, while in the pSico-CD8;LckCre mice recombination was detected in the thymus but not in other tissues (FIG. 5D and data not shown).

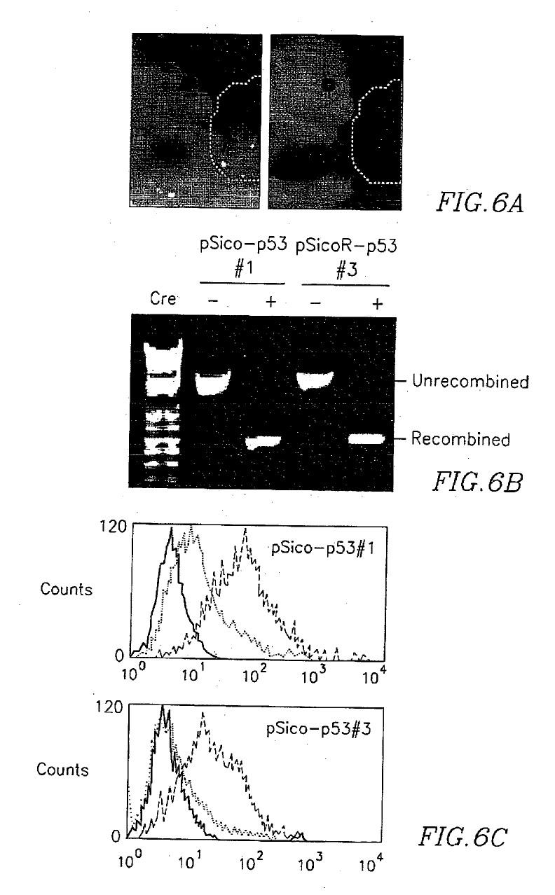

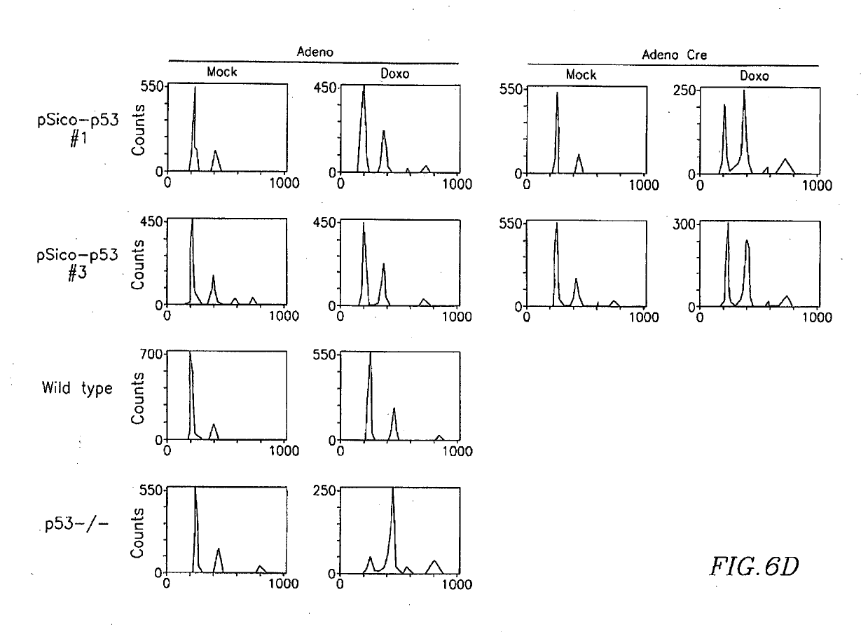

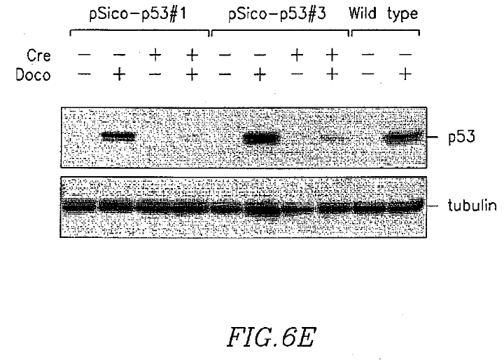

Tetraploid blastocyst complementation represents a faster alternative to diploid blastocyst injection because it allows the generation of entirely ES-derived mice without any passage through chimeras. In principle, this technology applied to pSico-infected ES cells would allow the generation of conditional knock-down mice in about 5-6 weeks (1 week for cloning the shRNA, 1-2 weeks for ES cells infection and clone selection and about two weeks for tetraploid blastocyst injection and gestation). To test this protocol directly, ES cells were infected with pSico-p53 and two different clones, pSico-p53#1 and pSico-p53#3, were injected into tetraploid blastocysts. As a rapid way to assess the inducibility of the p53 shRNA in ES cell-derived animals, midgestation embryos were recovered from two recipients females. Two apparently normal, GFP positive embryos were recovered, one each from ES clone pSico-p53 #1 and pSico-p53 #3 (FIG. 6A and data not shown). MEFs generated from these embryos were passaged once and infected with Ad or Ad-Cre. As expected, Cre expression induced significant recombination and loss of GFP expression (FIG. 6B, C). Importantly, in Ad-Cre-infected cells, p53 induction and cell cycle arrest following doxorubicin treatment were significantly inhibited compared to Ad-infected control cells (FIG. 6D, E).

Thus, the pSico and pSicoR vectors allowed for the conditional, Cre-mediated, RNA interference in mice.

1. A method of conditionally reducing expression of a coding sequence in a target cell, said method comprising contacting said target cell with a vector comprising:
i. An RNA Polymerase III promoter engineered to comprise a TATA-lox sequence;
ii. A transcriptional terminator sequence downstream of said TATA-lox sequence; and
iii. A second TATA-lox sequence upstream of an miRNA agent specific for said coding sequence, wherein said second TATA-lox sequence is downstream of said transcriptional terminator sequence;
wherein said target cell is capable of expressing a Cre recombinase and whereby, following Cre-mediated recombination, said miRNA agent is expressed and reduces expression of said coding sequence, thereby conditionally reducing expression of a coding sequence in a target cell.

2. The method according to claim 1, wherein said RNA Polymerase III promoter is a U6 promoter.

3. The method according to claim 1, wherein said cell is engineered to express a Cre recombinase.

4. The method according to claim 1, wherein said cell endogenously expresses a Cre recombinase.

5. The method according to claim 1, wherein said target cell is contacted with said vector in vivo, in vitro or ex-vivo.

6. The method according to claim 5, wherein said cell is in vivo, and said Cre recombinase is expressed at specific times during development.

7. The method according to claim 1, wherein said miRNA agent is an shRNA.

8. The method according to claim 7, wherein said shRNA specifically inactivates p53, nucleolar protein nucleophosmin (NPM) or DNA methyltransferase (DNMT-1) gene expression.

9. The method according to claim 1, wherein said TATA-lox sequence corresponds to, or is homologous to SEQ ID NO: 7.

10. The method according to claim 1, wherein said RNA Polymerase III promoter engineered to incorporate a TATA-lox sequence has a nucleotide sequence corresponding to, or homolgous to SEQ ID NO: 10.

11. The method according to claim 1, wherein said transcriptional terminator is upstream of a promoter operatively linked to a reporter gene.

12. A non-human animal with reduced expression of a coding sequence, wherein said reduced expression is produced according to the method of claim 1.

13. A mammalian cell with reduced expression of a coding sequence, wherein said reduced expression is produced in said cell according to the method of claim 1.

14. A method of conditionally expressing a coding sequence in a target cell, the method comprising contacting said target cell with a vector comprising:
i. An RNA Polymerase III promoter downstream of a loxP site;
ii. An miRNA agent specific for said coding sequence, operatively-linked thereto; and
iii. A loxP site downstream of said miRNA agent;
wherein said cell expresses said miRNA agent, thereby reducing expression of said coding sequence and whereby, following expression of said miRNA agent, Cre-mediated recombination is enabled in said target cell, such that said miRNA agent is no longer expressed, thereby being a method of conditionally expressing a coding sequence in a target cell.

15. The method according to claim 14, wherein said RNA Polymerase III promoter is a U6 promoter

16. The method according to claim 14, wherein said cell is engineered to express a Cre recombinase.

17. The method according to claim 14, wherein said cell endogenously expresses a Cre recombinase.

18. The method according to claim 14, wherein said target cell is contacted with said vector in vivo, in vitro or ex-vivo.

19. The method according to claim 18, wherein said cell is in vivo, and said Cre recombinase is expressed at specific times during development.

20. The method according to claim 14, wherein said miRNA agent is an shRNA.

21. The method according to claim 20, wherein said shRNA specifically inactivates p53, nucleolar protein nucleophosmin (NPM) or DNA methyltransferase (DNMT-1) gene expression.

22. A non-human animal with reactivated expression of a coding sequence, wherein said reactivated expression is produced according to the method of claim 14.

23. A mammalian cell with reactivated expression of a coding sequence, wherein said reactivated expression is produced according to the method of claim 14.

24. A vector comprising:
i. An RNA Polymerase III promoter engineered to comprise a TATA-lox sequence;
ii. A transcriptional terminator sequence downstream of said TATA-lox sequence; and
iii. A second TATA-lox sequence upstream of an miRNA agent specific for said coding sequence, wherein said second TATA-lox sequence is downstream of said transcriptional terminator sequence;

25. The vector of claim 24, wherein said RNA Polymerase III promoter is a U6 promoter

26. The vector of claim 24, wherein said miRNA agent is an shRNA.

27. The vector of claim 26, wherein said shRNA specifically inactivates p53, nucleolar protein nucleophosmin (NPM) or DNA methyltransferase (DNMT-1) gene expression.

28. The vector of claim 24, wherein said TATA-lox sequence corresponds to, or is homologous to SEQ ID NO: 7.

29. The vector of claim 24, wherein said RNA Polymerase III promoter engineered to incorporate a TATA-lox sequence has a nucleotide sequence corresponding to, or homolgous to SEQ ID NO: 10.

30. The vector of claim 24, wherein the backbone of said vector is derived from a lentivirus.

31. The vector of claim 24, further comprising a reporter gene.

32. The vector of claim 31, wherein said reporter gene is operatively linked to a promoter sequence, which is downstream of said transcriptional terminator.

33. A composition comprising the vector of claim 24.

34. A method of producing an animal genetically inactivated for a coding sequence, the method comprising:
a. contacting an embryonic stem cell with the vector of claim 24;
b. injecting the embryonic stem cell in (a) to a blastocyst of said animal; and
c. obtaining an animal in (b) expressing said vector
whereby, following Cre-mediated recombination in said animal, said miRNA agent is expressed and reduces expression of said coding sequence, thereby being a method of producing an animal genetically inactivated for a coding sequence.

35. A method of producing an animal genetically inactivated for a coding sequence, the method comprising:
a. contacting a single cell embryo of said animal with the vector of claim 24; and
b. obtaining an animal expressing said vector
whereby, following Cre-mediated recombination in said animal, said miRNA agent is expressed and reduces expression of said coding sequence, thereby being a method of producing an animal genetically inactivated for a coding sequence.

36. A method of identifying a gene product involved in carcinogenesis, the method comprising:
a. Obtaining the animal of claim 29 or 30, wherein said coding sequence is for a gene product which is putatively involved in carcinogenesis;
b. Maintaining the animal in (a) under conditions facilitating carinogenesis;
c. Initiating or enabling Cre-mediated recombination in the animal in (b); and
d. Identifying the inhibition or suppression of carcinogenesis in the animal in (c),
Wherein inhibition or suppression of carcinogenesis in said animal indicates said coding sequence is from a gene whose product is involved in carcinogenesis.

37. A vector comprising:
i. An RNA Polymerase III promoter downstream of a loxP site;
ii An miRNA agent specific for said coding sequence, operatively-linked thereto; and
iii. A loxP site downstream of said miRNA agent.

38. The vector of claim 37, wherein said RNA Polymerase III promoter is a U6 promoter

39. The vector of claim 37, wherein said RNAi agent is an shRNA

40. The vector of claim 39, wherein said shRNA specifically inactivates p53, nucleolar protein nucleophosmin (NPM) or DNA methyltransferase (DNMT-1) gene expression.

41. The vector of claim 37, wherein the backbone of said vector is derived from a lentivirus.

42. A composition comprising the vector of claim 37.

43. A method of producing an animal genetically reactivated for a coding sequence, the method comprising:
a. contacting an embryonic stem cell with the vector of claim 37;
b. injecting the embryonic stem cell in (a) to a blastocyst of said animal; and
c. obtaining an animal in (b) expressing said vector
whereby, following Cre-mediated recombination in said animal, said miRNA agent is no longer expressed and said coding sequence is expressed, thereby being a method of producing an animal genetically reactivated for a coding sequence.

44. A method of producing an animal genetically reactivated for a coding sequence, the method comprising:
a. contacting a single cell embryo of said animal with the vector of claim 31; and
b. obtaining an animal expressing said vector whereby, following Cre-mediated recombination in said animal, said miRNA agent is no longer expressed and said coding sequence is expressed, thereby being a method of producing an animal genetically reactivated for a coding sequence.

45. A method of identifying a tumor suppressor gene, the method comprising:
a. Obtaining the animal of claim 37 or 38, wherein said coding sequence is for a putative tumor suppressor;
b. Maintaining the animal in (a) under conditions promoting carcinogenesis;
c. Initiating or enabling Cre-mediated recombination the animal in (b) following carcinogenesis; and
d. Identifying inhibition or suppression of carcinogenesis in the animal in (c),
Wherein inhibition or suppression of carcinogenesis in said animal indicates said coding sequence is from a tumor suppressor gene.
